(19) United States
(12) Patent Application Publication
(10) Pub. No.: US 2017/0188688 A1
(43) Pub. Date: Jul. 6, 2017

(54) COSMETIC TREATMENT OF KERATIN FIBRES

(71) Applicant: L'OREAL, Paris (FR)

(72) Inventors: Henri SAMAIN, Bievres (FR); Franck GIRON, Lagny Sur Marne (FR)

(73) Assignee: L'OREAL, Paris (FR)

(21) Appl. No.: 15/315,337

(22) PCT Filed: May 29, 2015
(86) PCT No.: PCT/IB2015/054050

§ 371 Date: Nov. 30, 2016

(30) Foreign Application Priority Data
May 30, 2014 (FR) 1454924

(51) Int. Cl. A45D 40/26; A41G 5/02; A61Q 1/10; A45D 2/48; A61K 8/92; A61K 8/81

(52) U.S. Cl. CPC A45D 40/26; A61K 8/927; A61K 8/8111; A61Q 1/10; A45D 2/48; A41G 5/02; A61K 2800/43; A61K 2800/87

(57) ABSTRACT

The present invention relates to a process for cosmetic treatment of one or more keratin fibres, in particular human keratin fibres, especially one or more eyelashes or eyebrows, comprising the moulding of at least one cosmetic composition onto at least one part of said fibre(s), by means of one or more cavities (5) of a mould (2) into which said fibre(s) is (are) at least partially introduced.

The present invention relates to the cosmetic treatment of keratin fibres, in particular human keratin fibres, especially eyelashes or eyebrows.

It is sought to cosmetically treat keratin fibres in order to give them greater visibility and/or an original appearance.

Known solutions exist, such as the application of mascara and the use of additional fibres.

The first of these two solutions is effective for increasing eyelash visibility, but it is limited in terms of gain in visibility.

Attempts have been made to exceed this limit, in particular by increasing the amount of material deposited. If the material deposited is increased by means of a suitable brush, or by increasing the number of brushstrokes, a limit is quickly reached, due to the fact that, at each new deposit, almost as much material is removed as is deposited. Unattractive overloads and agglomerates are frequently created.

This solution is also limited by the number of possible aesthetic effects. For example, it is very difficult to obtain very smooth and therefore very glossy coatings.

The documents FR 2 936 420 A1, FR 2 923 381 A1, US 2007/286831 A1 and WO 2006/043544 A1 disclose application of mascara with mascara brushes.

The second solution consists in sticking additional fibres, in particular false eyelashes, onto the eyelashes.

A first technique consists in sticking false eyelash fringes onto the eyelid: they are difficult to put in place and the result is not very natural. Furthermore, persistence is low, being limited to approximately one day. Such false eyelash fringes, and also the processes for producing them, are described in patents U.S. Pat. No. 2,421,432 and U.S. Pat. No. 3,559,657.

Another technique consists in sticking false eyelashes onto the eyelashes one by one: the effect is not very visible, is longer lasting but lengthy to perform, expensive and can only be carried out by an individual working in the field.

A means for cosmetically treating eyelashes in order to obtain effects of high volume and/or great length, without the risk of eyelashes sticking together, is therefore sought. A means for creating new effects on keratin fibres, such as smooth and glossy coating effects, all with a process that is easy to use, is also sought.

The invention is directed towards satisfying this need, and a subject matter thereof is a process for cosmetic treatment of one or more keratin fibres, in particular human keratin fibres, especially one or more eyelashes or eyebrows, comprising the moulding of at least one cosmetic composition onto at least one part of said fibre(s), by means of one or more cavities of a mould into which said fibre(s) is (are) at least partially introduced.

Thus, instead of depositing a product on the keratin fibres while forcing it to spread thereon, for example using a brush, or of sticking on additional fibres, in particular false eyelashes, a composition is moulded around the keratin fibres.

This makes it possible to control the shape of the deposit of composition on the treated fibres and to obtain, whatever the dexterity of the individual, a reproducible result. The invention also makes it possible to generate new inaccessible shapes, to exceed the length of the keratin fibres or the thickness of the usual deposits, to produce inaccessible colour and glossy effects, and to easily integrate additional fibres, such as false eyelashes.

The surface finish of the mould can be chosen so as to confer on the deposit a smooth external surface and a glossy appearance if desired.

A subject of the invention is also an assembly for carrying out the process according to the invention, comprising at least one cosmetic composition for cosmetic treatment of keratin fibres, in particular human keratin fibres, especially eyelashes or eyebrows, and a device comprising a mould, the mould comprising at least one cavity, in particular at least two, preferably at least six, more preferably at least ten cavities, into which or each of which at least one of said fibres can be at least partially introduced, in such a way that the composition is moulded onto at least one part of said fibre(s) present in the cavity or cavities. The cavity or cavities may be formed by bringing two jaws together, in particular according to a translational movement of one of the two jaws with respect to the other.

Unless otherwise mentioned, the parameters according to the invention are given at ambient temperature and pressure, i.e. respectively at 25° C. and at 1 atmosphere.

I. Keratin Fibres

The keratin fibres to which the process according to the invention applies are preferably human keratin fibres, in particular eyelashes or eyebrows, more preferably eyelashes.

The keratin fibres may be hair. It is thus possible to treat the hair, in particular on a part of the length thereof, for instance the roots, in order to increase the rigidity thereof, and/or the ends in order to improve the appearance thereof.

It is possible to post-treat the keratin fibres, moulded according to the invention, with other products, for example mascara, or by contact with a hot surface.

Each of said fibres may be at least partially introduced into a respective cavity. At least two of said fibres may be at least partially introduced into the same cavity. At least one cavity may contain just one fibre.

The composition may be deposited on at least three quarters of the length of at least one of said fibres, better still on at least three quarters of the length of each of said fibres.

The length of a fibre is measured from the surface of the skin up to its free end when the fibre is placed flat.

The composition may be deposited on just one part of the length of at least one of said fibres, better still on just one part of the length of each of said fibres.

II. Mould

The moulding is carried out in situ in the mould cavity or cavities, and the moulding allows the composition to be shaped. Thus, the composition can fit the shape of the mould cavity or cavities.

The mould may comprise one or more imprints, which each at least partially define a mould cavity. Preferably, the mould is not limited to a single cavity.

The mould may comprise at least two parts which each comprise one or more imprints, the imprint(s) of one of the parts being placed opposite the imprint(s) of at least one other part, so as to form the mould cavity or cavities when the parts of the mould are brought together, each of the imprints being in particular of rounded cross section, notably of circular arc, elliptical arc or parabolic cross section, in particular of semicircular cross section.

The mould may comprise at least two parts, one part comprising one or more imprints, each of the imprints being in particular of rounded cross section, notably of circular arc, elliptical arc or parabolic cross section, in particular of semicircular cross section, the other part being devoid of imprint, so as to form the cavity or cavities of the mould when the parts of the mould are brought together. Such a mould has the advantage of not requiring a great deal of precision when placing the two parts of the mould opposite one another so as to form the cavities.

The imprint(s) may be of rounded cross section to fit the keratin fibre(s) shape.

The imprints may be parallel to one another.

The imprints may be arranged along only one part of the mould, in particular in the front of said mould, i.e. on the side where the keratin fibres, in particular human keratin fibres, are introduced.

Each imprint may have a depth of between 75 μm and 1.5 mm.

The mould may remain motionless relative to the keratin fibres during the moulding of the fibres in its cavity or cavities.

The mould preferably does not move along the keratin fibres during the moulding of the fibres in its cavity or cavities.

The mould may or may not be disposable. In particular, it is disposable.

The surface finish of the mould may be smooth so as to confer on the composition once moulded a smooth external surface and a glossy appearance.

There can not be any relative speed between the keratin fibres and the cavities during the moulding, for example for a period required for the hardening or the drying of the composition, for example a period of 10 to 60 seconds.

III. Mould Cavity

The volume of the cavity or cavities may exceed by at least a factor of 2, better still a factor of 10 to 100, the volume of the part of the keratin fibres, in particular human keratin fibres, filling it or them.

The volume of each cavity may range between 0.08 and 220 mm³.

The cavity or cavities may each have a circular, semicircular, oval or polygonal cross section, which may or may not be constant when moving along the cavity.

The cavity or cavities may each have an approximately cylindrical shape.

The cavity or cavities may each have a decreasing cross section so as to give the fibre(s), once moulded by the composition, a tapered appearance and to facilitate, where appropriate, the demoulding of the fibres by pulling them away.

The cavity or cavities may each have an approximately cylindrical shape on one part of their length and an enlarged, in particular spherical, shape on another part of their length, in particular with a view to moulding a head, for example in the shape of a portion of a sphere, on at least one part of the keratin fibre(s), in particular human keratin fibre(s).

The cavity or cavities may each have a length of between 5 and 30 mm and a width, in particular a diameter, of between 150 μm and 3 mm. In the case of a length of 30 mm and a diameter of 3 mm, the volume may be increased by a factor of 50.

Each cavity may have an elongated shape with a curvilinear longitudinal axis which approximately reproduces or which increases the curvature of the keratin fibre(s), in particular human keratin fibre(s), introduced, in particular the eyelashes. Thus, the moulding enables to increase keratin fibres visibility while underlining their curvature.

The radius of curvature of the cavity or cavities may be higher than 15 mm. In particular, it can range between 15 and 25 mm.

Each cavity may, on the contrary, have an elongated shape which does not reproduce the curvature of the keratin fibre(s), in particular human keratin fibre(s), introduced, having in particular a rectilinear or broken-line longitudinal axis. The cavity or cavities may extend in all directions, in particular transversely to the eyelashes. The cavity or cavities may form patterns of any type, such as waves or grids.

Each cavity may have an elongated shape, with a curvilinear or rectilinear longitudinal axis, and the cavities may have non-parallel respective longitudinal axes.

When the cavity or cavities have an elongated shape, the keratin fibres are preferably introduced into the cavity or cavities along their longitudinal axes.

Although cavities of elongated shape make it possible to perform a more conventional cosmetic treatment of keratin fibres, in particular human keratin fibres, new effects can be obtained with cavities of non-elongated shape. For example, the cavity or cavities may each have an approximately spherical or polyhedral shape, in particular spherical shape, having a diameter of between 0.4 mm and 5 mm, so as to form balls at the end of the keratin fibres, in particular human keratin fibres, for example.

Each cavity is preferably closed laterally, so that the composition does not escape laterally during the moulding.

Each cavity may have an elongated shape and be closed at at least one of its longitudinal ends.

Each cavity may constitute a space which is substantially entirely closed, notably entirely closed, in the absence of keratin fibre, in particular human keratin fibre. In this case, one part of each keratin fibre, in particular human keratin fibre, at least partially introduced into a cavity may be gripped between the two parts of the mould that are brought together, while another part of the keratin fibre, in particular human keratin fibre, extends into the cavity. The cavity may be closed on the side where the keratin fibre(s), in particular human keratin fibre(s), is (are) introduced, via a flexible wall, which can deform, preferably elastically, so as to allow the keratin fibre(s), in particular human keratin fibre(s), to pass into the cavity.

As a variant, each cavity may constitute a semi-open space, in the absence of keratin fibre, in particular human keratin fibre. In particular, each cavity can delimit a space which is closed with the exception of one end via which the keratin fibre(s), in particular human keratin fibre(s), which is (are) at least partially introduced into the cavity communicate(s) with the exterior. Thus, each cavity may open to the exterior, on the side where the keratin fibre(s), in particular human keratin fibre(s), is (are) introduced, via an opening through which the keratin fibre(s) is (are) introduced.

The free end of the keratin fibre(s) introduced into the cavity or cavities preferably do not exceed the cavity or cavities.

At least two of the cavities may be not connected. All of the cavities may be not connected.

The cavities may or may not be parallel to one another.

The cavities may be placed at regular intervals, for example according to a step of 1 to 2 mm, centre to centre.

The number of cavities may be greater than or equal to one, in particular greater than or equal to two, preferably greater than or equal to six and more preferably greater than or equal to ten.

IV. Jaws

The cavity or cavities may be formed by bringing two jaws together, in particular according to a translational and/or rotational movement of one of the jaws with respect to the other.

The two jaws can be moved apart at the moment the keratin fibres, in particular human keratin fibres, are introduced and closed again so as to form the cavities into which the keratin fibres, in particular human keratin fibres, are introduced.

The jaws may carry or integrate the mould.

The jaws may be curved, in particular about a geometric axis perpendicular to the longitudinal axis of the mould cavities.

A system where one or both of the jaws slide(s) on columns can be used to move the two jaws apart at the moment the keratin fibres, in particular human keratin fibres, are introduced and to close them again so as to form the cavities.

Use may also be made of a system where the jaws are connected by a hinge.

Use may also be made of a system where the jaws are not connected, and are optionally associated with one another by a guide system.

The two jaws preferably fit together sufficiently well for the composition present in the cavities not to escape.

The jaws may comprise an elastomer material, for example in order to absorb a deformation of the mould.

V. Mould Deformation

It may be advantageous to deform the mould with the keratin fibres in place in order to reduce the volume of the cavities, and for example to force the composition to be distributed around the fibres introduced into the cavities.

The mould may comprise a flexible material, in particular a plastic, especially an elastomeric material.

An extensible, in particular elastomeric, mould makes it possible to compress the composition in order to avoid problems of dead volume and/or to force better integration of the fibres into the composition. The mould may be entirely made of flexible material, in particular plastic, especially elastomeric material.

The mould may comprise flexible parts and non-flexible parts. The mould may in particular be surface-covered with flexible, in particular elastomeric, material, for example over a thickness ranging from 0.5 to 2 mm.

The deformation of the mould may be carried out by compression, for example mechanical compression with fingers, or be pneumatic or hydraulic, or by suction. For example, by pulling a trigger, an overpressure can be created between one jaw and the mould, which has the effect of compressing the two parts of the mould onto one another and preventing dead zones.

VI. Demoulding

The composition can be demoulded, and extracted from the mould, preferably without losing its cohesion around the keratin fibres, in particular human keratin fibres, and while retaining the surface finish conferred by the mould.

Demoulding of the composition without deterioration is desired.

The mould may be extensible, in particular elastomeric, in order to facilitate demoulding.

The cavity or cavities of the mould may have a non-stick coating or may have undergone a treatment aimed at conferring non-stick properties. Thus, the mould may comprise at the surface a low-adhesion material, in particular of silicone or PTFE type. A layer of a non-stick product, in particular an oil, a silicone, a PTFE powder or boron nitride, may also be applied to the mould cavity.

The device may comprise an automatic or non-automatic demoulding system in order to act on the mould and/or the composition contained in the cavity or cavities in order to facilitate the separation of the composition-coated fibres from the mould. This demoulding system may comprise a set of small blades or other reliefs which deform the mould by being compressed against said mould. The deformation of the mould can take place at the level of the cavities, thereby facilitating the ejection of the moulded material.

Bridges of composition may connect several cavities to one another after moulding, in an unwanted manner. The device may comprise blades or other reliefs which are used to cut bridges of composition between at least two cavities after moulding. These blades or other reliefs may act by fitting over the bridges between the cavities. These blades may be added to the mould.

The blades or other reliefs which are used to cut the bridges of composition may be carried by one of the jaws. In this case, the other jaw may have a planar surface from the viewpoint of the blade or other relief or a groove into which the blade or other relief fits. The blades or other reliefs may further be placed on the two jaws, in such a way that one blade or other relief of one of the jaws fits over one blade or other relief of the other jaw.

The demoulding of the moulded composition is preferably carried out mechanically, in particular by deformation of the mould, by moving the two jaws away from one another and/or by pulling the keratin fibres, in particular human keratin fibres, out of the mould.

The demoulding of the composition may be carried out, where appropriate, by pulling at one end of one part of the mould in order to separate it, from the other part, in the way in which two sheets adhering to one another are separated by peeling. The demoulding of the moulded composition may further, according to one variant, be at least partly carried out physicochemically, in particular by at least partially dissolving, with a solvent, the mould or a film-coating present inside the mould, between said mould and the composition moulded onto the keratin fibres, in particular human keratin fibres.

VII. Provision of the Composition

At least one part of the composition, in particular the entire composition, may be deposited on at least one of said fibres, better still on each of said fibres, before they are introduced into the mould. In this case, the closing of the mould can distribute the composition in the cavities and, where appropriate, drive the excess composition out of the mould.

At least one part of the composition, in particular the entire composition, may be initially present in the mould before introduction of the fibre(s) into the mould. This can facilitate the metering of the composition introduced into the mould cavity or cavities. The composition may be contained with the mould in sealed packaging.

At least one part of the composition, in particular the entire composition, may be injected into the mould, via at least one injection channel.

The mould may comprise imprints in which at least one part of the composition, in particular the entire composition, is present before introduction of the keratin fibre(s) into the mould. The imprints define the mould cavities when said mould is closed.

The mould may comprise at least two parts each comprising imprints, at least one part of the composition, in particular the entire composition, being present in at least one part, in particular in all the parts, of the mould, before introduction of the keratin fibre(s) into the mould. The composition for example entirely fills the imprints and is present only in the imprints, before closing of the mould.

The mould may also be prefilled with an excess of the composition so that the material stands higher than the cavity, for example by being slightly curved. In this way, any absence of product around the eyelash is avoided and the formation of a perfect cast is ensured.

VIII. Automatism

The device may be automated. A single triggering may make it possible to perform a series of operations, for example filling the cavities with the moulding composition, and an action exerted on the mould so as to deform it in order to perform the demoulding. The closing of the mould may also be automatic.

IX. Device

The device may comprise at least one heating element which serves to increase the temperature of the composition. The increase in temperature can induce a change of state of the composition, in particular a change from the solid state to the liquid state by melting, or the solidification of the composition, or else an increase in the reactivity of the composition.

During the temperature increase, the composition may be present in the mould or outside the mould. The composition may be present outside the mould during the increase in its temperature and may be injected in the liquid state into the mould, in particular via at least one injection channel.

The heating may be automatically triggered upon closure of the mould. As a variant, the heating may be triggered before closure of the mould and a visual and/or sound indicator may signal to the user that the desired temperature for closing the mould and/or putting the fibres in place has been reached.

The device may comprise at least one evaporation system which serves to evaporate a solvent. This may be a heating element and/or a suitable ventilation.

The device may comprise at least one system for admitting material, which serves to introduce a part or all of the composition into the mould cavity or cavities, in particular comprising one or more injection channels communicating with one or more mould cavities. The composition is, for example, contained in a reservoir, and a piston or a pump makes it possible to force it to flow into the mould cavity or cavities.

The device may comprise at least one light element, in particular IR, UV or visible light element, or microwave element, which serves in particular to increase the temperature of the composition, in particular by absorption of the light or microwave radiation by the cosmetic composition.

The jaws may be closed manually.

The device may be in the form of a clamp comprising at least one housing into which at least one of the fingers of one hand, for example the thumb or the index finger, can be introduced so as to move the two jaws apart, in order to make it possible to introduce the keratin fibres, in particular human keratin fibres, and to close them on said fibres.

The device may be in the form of a clamp comprising two housings into which two of the fingers of one hand, for example the thumb and the index finger, can be introduced so as to move the two jaws apart, in order to make it possible to introduce the keratin fibres, in particular human keratin fibres, and to close them on said fibres. Each of these housings may be removable or non-removable. It may or may not be possible for each of these housings to be oriented rotationally.

X. Cosmetic Composition

The composition is a cosmetic composition. It can be removed from the keratin fibres, in particular human keratin fibres, by conventional makeup-removing processes, for example by applying water, in particular warm water.

A. Composition Having a Melting Point of Between 40° C. and 120° C.

According to a first embodiment variant, the cosmetic composition of the process or of the assembly according to the invention has a melting point of between 40° C. and 120° C., and comprises at least 15% by weight of meltable compound(s), relative to the total weight of the composition.

In particular, such a composition is brought to a temperature greater than or equal to its melting point.

Thus, the present invention relates to a process for cosmetic treatment of one or more keratin fibres, in particular of one or more eyelashes or eyebrows, comprising at least the step consisting in moulding a cosmetic composition having a melting point of between 40° C. and 120° C., and comprising at least 15% by weight of meltable compound(s), relative to the total weight of the composition, onto at least one part of said fibres, by means of one or more cavities (5), of a mould (2), into which said fibres are at least partially introduced.

According to another of its aspects, the invention relates to a cosmetic assembly, of use for the treatment of keratin fibres, in particular the eyelashes and the eyebrows, comprising:
a cosmetic composition having a melting point of between 40° C. and 120° C., and comprising at least 15% by weight of meltable compound(s), relative to the total weight of the composition; and
a device (1) comprising a mould (2), the mould (2) comprising at least one cavity (5), and preferably several cavities (5), into which or each of which at least one of said fibres can be at least partially introduced for the purposes of moulding said composition onto at least one part of said fibre(s) present in the cavity or cavities (5).

According to yet another of its aspects, the present invention relates to the use of a cosmetic composition having a melting point of between 40° C. and 120° C., and comprising at least 15% by weight of meltable compound(s), relative to the total weight of the composition, for application thereof as a moulding material in at least one cavity of a mould, for the purposes of depositing on keratin fibres, in particular eyelashes and eyebrows, via the application of said mould to said fibres.

In particular, the cosmetic composition has a melting point of between 40° C. and 100° C., preferably between 45° C. and 85° C.

For the purposes of the invention, the melting point corresponds to the temperature of the most endothermic peak observed in thermal analysis (DSC) as described in the standard ISO 11357-3; 1999. The melting point may be measured using a differential scanning calorimeter (DSC), for example the calorimeter sold under the name “DSC Q2000” by the company TA Instruments.

The measuring protocol is as follows:

A 5 mg sample placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature rise ranging from −20° C. to 100° C. at a heating rate of 5° C./minute. During the second temperature rise, the following parameters are measured:
the melting point (T_{f}) of the sample, which corresponds to the temperature value of the most endothermic peak of the observed melting curve, representing the variation in the difference in power absorbed as a function of the temperature;
Δhf: the fusion enthalpy of the sample, corresponding to the integral of the entire obtained melting curve. Said fusion enthalpy is the amount of energy required to make the composition pass from the solid state to the fluid state. It is expressed in J/g.

Such a cosmetic composition may be fluid or solid at 25° C. and at 1 atm.

It may be aqueous or anhydrous, and preferably anhydrous.

It may have a solids content of greater than or equal to 42% by weight, in particular greater than or equal to 45% by weight, or even greater than or equal to 48% by weight, and preferentially greater than or equal to 50% by weight, relative to the total weight of the composition.

For the purposes of the present invention, the term “solids content” denotes the content of non-volatile matter.

The solids content (abbreviated as SC) of a composition according to the invention is measured using a “Halogen Moisture Analyser HR 73” commercial halogen desiccator from Mettler Toledo. The measurement is performed on the basis of the weight loss of a sample dried by halogen heating, and thus represents the percentage of residual matter once the water and the volatile matter have evaporated off.

This technique is in particular described in the documentation of the apparatus supplied by Mettler Toledo.

The measuring protocol is as follows:

Approximately 2 g of the composition, referred to hereinbelow as the sample, are spread out on a metal crucible, which is placed in the halogen desiccator mentioned above. The sample is then subjected to a temperature of 120° C. until a constant weight is obtained. The wet mass of the sample, corresponding to its initial mass, and the dry mass of the sample, corresponding to its mass after halogen heating, are measured using a precision balance.

The experimental error associated with the measurement is of the order of plus or minus 2%.

The solids content is calculated in the following manner:

Solids content (expressed as % by weight)=100×(dry mass/wet mass).

According to a first embodiment, the cosmetic composition is fluid at ambient temperature.

For the purposes of the invention, the description “fluid” is intended to characterize the fact that a composition according to the invention is not solid. In other words, it manifests a fluidity sufficient to have flow properties. A composition of mascara type is, for example, representative of this type of fluidity.

In particular, the composition according to the invention may advantageously have a viscosity of less than 100 Pa·s, preferably between OA Pa·s and 50 Pa·s, and better still between 1 Pa·s and 30 Pa·s, at ambient temperature and pressure, the viscosity being in particular measured using a Rheomat RM100®.

Such a composition may be aqueous or anhydrous.

It may be in the form of oil-in-water emulsions, as is the case for wax-in-water emulsions. It may also be in the form of a dispersion of wax particles in an anhydrous medium, as is the case with a dispersion of wax in isododecane.

According to this first embodiment, the cosmetic composition may comprise from 15% to 60% by weight, preferably from 18% to 55% by weight and even better still from 20% to 50% by weight of meltable compound(s), relative to the total weight of the composition.

According to a second embodiment, the cosmetic composition is solid at ambient temperature.

For the purposes of the invention, the description “solid” is intended to characterize the fact that a composition according to the invention is not liquid. In other words, it manifests a rigidity sufficient to not have flow properties.

The composition may thus advantageously have a viscosity greater than 1000 Pa·s, preferably greater than 10 000 Pa·s, at ambient temperature and pressure.

Such a composition may be aqueous, in particular in the form of an emulsion of the meltable compound(s) in water, or anhydrous. In particular, it may be in the form of a dispersion of the meltable compound(s) in an organic, preferably volatile, preferentially hydrocarbon-based, solvent.

Such a cosmetic composition is preferably anhydrous.

According to this second embodiment, the cosmetic composition comprises from 40% to 100% by weight, preferably from 60% to 100% by weight and even better still from 80% to 100% by weight of meltable compound(s), relative to the total weight of the composition.

A composition according to the invention may be subjected to heating means before and/or during the application.

These heating means are suitable for melting at least one part of the meltable compound(s) of the cosmetic composition.

The composition may be locally heated to a temperature greater than or equal to 45° C., or even greater than or equal to 50° C., or else greater than or equal to 55° C.

The temperature to which at least part of the composition is heated may be inclusively between 45° C. and 120° C., better still between 45° C. and 85° C.

The temperature may be measured, for example, at the surface using an infrared pyrometer, for example a Fluke® brand machine.

Only the heated composition can come into contact with the keratin fibres, for example the eyelashes, during the application.

It is understood that the temperature of the cosmetic composition must not lead to a risk of burning at the time of application.

Thus, when the composition is heated before application, a waiting time between the moment at which the composition is heated and the application to the keratin materials may optionally be necessary.

According to one embodiment variant, the composition is heated simultaneously with its application to the keratin fibres.

According to another embodiment variant, the composition is heated before and during its application to the keratin fibres.

The total heat of fusion of the composition is the heat consumed by the composition between −20° C. and 120° C. The total heat of fusion of the composition is equal to the area under the curve of the thermogram obtained using a differential scanning calorimeter (DSC), such as the calorimeter sold under the name MDSC 2920 by the company TA Instrument, with a temperature rise of 5° C. or 10° C. per minute, according to standard ISO 11357-3:1999.

The measuring protocol is as follows:

A 5 mg sample of composition is placed in a crucible and then subjected to a first temperature rise ranging from −20° C. to 120° C., at a heating rate of 10° C./minute, and is then cooled from 120° C. to −20° C. at a cooling rate of 10° C./minute. The sample is maintained at −20° C. for 5 minutes and finally subjected to a second temperature rise ranging from −20° C. to 100° C. at a heating rate of 5° C./minute.

During the second temperature rise, the variation in the difference in power absorbed by an empty crucible and by the crucible containing the sample of the composition is measured as a function of the temperature. The melting point of the compound is the temperature value corresponding to the top of the peak of the curve representing the variation in the difference in power absorbed as a function of the temperature.

The heat of fusion of the composition consumed at the temperature Tc is the amount of energy Δh required to make the composition pass from the solid or very viscous state at −20° C. to the state of the composition at the temperature Tc. It is expressed in J/g.

According to one embodiment of the invention, the cosmetic composition is chosen such that, when said composition is heated to the temperature Tc, the ratio of the heat consumed between −20° C. and Tc by the composition to the total heat consumed Δh between −20° C. and 120° C. is greater than 0.4.

This relationship is confirmed, for example, for a temperature Tc of the composition of between 45° C. and 85° C.

The choice of the temperature Tc to which the composition is brought by the heating means may thus be made so that said ratio is greater than or equal to 0.4, for example greater than 0.5. In other words, heating is performed to a temperature such that the ratio of the heat supplied to heat the sample of composition to the temperature Tc to the total heat is greater than or equal to 0.4, such a parameter being measured according to the DSC protocol described above.

The composition in accordance with the invention is capable of passing from a solid state to an at least partially liquid or preferably even totally liquid state, and of doing so reversibly.

As mentioned above, a composition according to the invention comprises a content of meltable compound(s) of greater than 15% by weight, relative to the total weight of the composition. Preferably, it may have a content of meltable compound(s) ranging from 15% to 100%, better still from 20% to 95% by weight, relative to the total weight of the composition.

For the purposes of the invention, the meltable compound(s) advantageously has (have) a melting point of between 40° C. and 120° C.

Thus, preferably, a cosmetic assembly, of use for the treatment of keratin fibres, comprises a cosmetic composition having a melting point of between 40° C. and 120° C., and comprises at least 15% by weight of meltable compound(s) having a melting point of between 40° C. and 120° C., relative to the total weight of the composition; and a device comprising a mould, the mould comprising at least one cavity, into which or each of which at least one of said fibres can be at least partially introduced, for the purposes of moulding said composition onto at least one part of said fibre(s) present in the cavity or cavities.

Preferably, this (these) compound(s) can be chosen from thermoplastic polymers, waxes, semi-crystalline polymers, and mixtures thereof.

Thus, according to one particular embodiment, said meltable compound(s) may have crystallizable chains.

In this embodiment, the cosmetic composition is then heated to a temperature Tc such that at least one part of the crystallizable chains of the meltable compounds) is at least partially, or even totally, melted. The solid/liquid change of state is thus at least partly due to the melting of a crystalline part of the meltable compound(s).

Preferably, the meltable compound(s) according to the invention are not in the form of a particulate dispersion in a solvent medium.

Thermoplastic Polymer

For the purposes of the present invention, the term “thermoplastic polymer” is intended to mean a polymer which softens when hot and which can be moulded while retaining its shape after cooling.

The thermoplastic polymers that can be used in the context of the present invention are any polymer or copolymer or any blend of polymers and/or copolymers having the property of being thermoplastic.

Among the thermoplastic polymers, mention may in particular be made of polyethylene, polystyrene, polyamides, polyvinyl chloride, polyethylene terephthalate, and mixtures thereof.

Mention may also be made of aliphatic polyesters, and in particular polyhydroxyalkanoates (PHAs), such as poly-3-hydroxybutyrate (PHB), polyhydroxyvalerate (PHV) or polyhydroxyhexanoate (PITH), polylactic acids (PLAs), polybutylene succinates (PBSs), polycaprolactones (PCLs), polyanhydrides, polyvinyl alcohols, and derivatives thereof, acetate esters, such as acetate/polyvinyl (PVAc) copolymer, starch derivatives, polysaccharides, including in particular cellulose derivatives such as cellulose esters, and derivatives thereof, in particular celluloids or cellulose ethers, and mixtures thereof.

In particular, among the cellulose esters, mention may be made of cellulose acetate, cellulose triacetate, cellulose propionate, cellulose acetate propionate, cellulose acetate butyrate, and cellulose sulfate, and mixtures thereof.

Among the cellulose ethers, mention may in particular be made of methylcellulose, ethylcellulose, ethylmethylcellulose, hydroxyethylcellulose, hydroxypropylcellulose (HPC), hydroxyethylmethylcellulose, hydroxypropylmethyl cellulose (HPMC), ethylhydroxyethylcellulose, carboxymethylcellulose (CMC), and mixtures thereof.

Among the acetate esters, mention may in particular be made of acetate/polyvinyl copolymers, including in particular ethylene-vinyl acetate (EVA) and derivatives thereof. For example, mention may be made of EVA/ethylcellulose or EVA/starch copolymers.

As thermoplastic polymer quite particularly suitable for a composition according to the invention, mention may preferably be made of ethylene-vinyl acetate (EVA), in particular sold under the name Evatane 28-800 by the company Arkema.

In a cosmetic composition according to the invention, use may in particular be made of thermoplastic polymers formulated in a mixture, such as the mixture of ethylene-vinyl acetate and paraffin sold under the name Cool Bind 34-1300° by the company National Starch.

Preferably, the film-forming polymers under consideration in the context of the present invention are distinct from particulate dispersions of latex type.

Wax

The term “wax” is intended to mean in general a lipophilic compound that is solid at ambient temperature (25° C.), with a reversible solid/liquid change in state, having a melting point of greater than or equal to 30° C., which may be up to 200° C. and in particular up to 120° C.

For the purposes of the invention, the melting point corresponds to the temperature of the most endothermic peak observed in thermal analysis (DSC) as described in the standard ISO 11357-3; 1999. The melting point of the wax may be measured using a differential scanning calorimeter (DSC), for example the calorimeter sold under the name MDSC 2920 by the company TA Instruments.

The measuring protocol is as follows:

A 5 mg sample of wax placed in a crucible is subjected to a first temperature rise ranging from −20° C. to 100° C., at a heating rate of 10° C./minute, is then cooled from 100° C. to −20° C. at a cooling rate of 10° C./minute and is finally subjected to a second temperature rise ranging from −20° C. to 100° C. at a heating rate of 5° C./minute. During the second temperature rise, the variation in the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

The waxes that may be used in the compositions according to the invention are chosen from waxes that are solid at ambient temperature of animal, vegetable, mineral or synthetic origin, and mixtures thereof.

The waxes, for the purposes of the invention, may be those used generally in the cosmetic or dermatological fields. They may in particular be polar or apolar, and hydrocarbon-based, silicone and/or fluoro waxes, optionally comprising ester or hydroxyl functions. They may also be of natural or synthetic origin.

a) Apolar Waxes

For the purposes of the present invention, the term “apolar wax” is intended to mean a wax of which the solubility parameter at 25° C. as defined below, δₐ, is equal to 0 (J/cm³)^{1/2}.

The definition and calculation of the solubility parameters in the Hansen three-−dimensional solubility space are described in the article by C. M. Hansen: The three-dimensional solubility parameters, J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δₐ is determined by the equation: δₐ=(δₚ²+δₕ²)^{1/2}.

The parameters δₚ, δₕ, δ_{D} and δₐ are expressed in (J/cm³)^{1/2}.

The apolar waxes are in particular hydrocarbon-based waxes constituted solely of carbon and hydrogen atoms, and free of heteroatoms such as N, O, Si and P.

The apolar waxes are chosen from microcrystalline waxes, paraffin waxes, ozokerite and polyethylene waxes, and mixtures thereof.

An ozokerite that may be mentioned is Ozokerite Wax SP 1020 P.

As microcrystalline waxes that may be used, mention may be made of Multiwax W 445® sold by the company Sonneborn, and Microwax HW® and Base Wax 30540® sold by the company Paramelt, and Cerewax No. 3 sold by the company Baerlocher.

As microwaxes that may be used in the compositions according to the invention as apolar wax, mention may be made in particular of polyethylene microwaxes such as those sold under the names Micropoly 200®, 220®, 220L® and 250S® by the company Micro Powders.

Polyethylene waxes that may be mentioned include Performalene 500-L Polyethylene and Performalene 400 Polyethylene sold by New Phase Technologies, and Asensa® SC 211 sold by the company Honeywell.

b) Polar Wax

For the purposes of the present invention, the term “polar wax” is intended to mean a wax of which the solubility parameter at 25° C., δa, is other than 0 (J/cm³)^{1/2}.

In particular, the term “polar wax” is intended to mean a wax of which the chemical structure is formed essentially from, or even consists of, carbon and hydrogen atoms, and comprising at least one highly electronegative heteroatom such as an oxygen, nitrogen, silicon or phosphorus atom.

The polar waxes may in particular be hydrocarbon-based, fluoro or silicone waxes.

Preferentially, the polar waxes may be hydrocarbon-based waxes.

The term “hydrocarbon-based wax” is intended to mean a wax formed essentially from, or even constituted of, carbon and hydrogen atoms, and optionally oxygen and nitrogen atoms, and that does not contain any silicon or fluorine atoms. It may contain alcohol, ester, ether, carboxylic acid, amine and/or amide groups.

According to the invention, the term “ester wax” is intended to mean a wax comprising at least one ester function. According to the invention, the term “alcohol wax” is intended to mean a wax comprising at least one alcohol function, i.e. comprising at least one free hydroxyl (OH) group.

In particular, use may be made, as polar waxes, of those chosen from:
i) waxes of formula R₁COOR₂ in which R₁ and R₂ represent linear, branched or cyclic aliphatic chains in which the number of atoms ranges from 10 to 50, which may contain a heteroatom such as O, N or P and whose melting point ranges from 25 to 120° C.;
ii) bis(1,1,1-trimethylolpropane) tetrastearate, sold under the name Hest 2T-4S® by the company Heterene;
iii) diester waxes of a dicarboxylic acid of general formula R³—(—OCO—R⁴—COO—R⁵), in which R³ and R⁵ are identical or different, preferably identical, and represent a C₄-C₃₀ alkyl group (alkyl group comprising from 4 to 30 carbon atoms) and R⁴ represents a linear or branched C₄-C₃₀ aliphatic group (alkyl group comprising from 4 to 30 carbon atoms) which may or may not comprise one or more unsaturations and which is preferably linear and unsaturated;
iv) mention may also be made of the waxes obtained by catalytic hydrogenation of animal or vegetable oils having linear or branched C₈-C₃₂ fatty chains, for example such as hydrogenated jojoba oil, hydrogenated sunflower oil, hydrogenated castor oil, hydrogenated coconut oil, and also the waxes obtained by hydrogenation of castor oil esterified with cetyl alcohol;
v) beeswax, synthetic beeswax, polyglycerolated beeswax, carnauba wax, candelilla wax, oxypropylenated lanolin wax, rice bran wax, ouricury wax, esparto grass wax, cork fibre wax, sugar cane wax, Japan wax, sumach wax, montan wax, orange wax, laurel wax, hydrogenated jojoba wax, sunflower wax, lemon wax, olive wax or berry wax.

According to another embodiment, the polar wax may be an alcohol wax. According to the invention, the term “alcohol wax” is intended to mean a wax comprising at least one alcohol function, i.e. comprising at least one free hydroxyl (OH) group. Alcohol waxes that may be mentioned include for example the C₃₀₋₅₀ alcohol wax Performacol® 550 Alcohol sold by the company New Phase Technologies, stearyl alcohol and cetyl alcohol.

It is also possible to use silicone waxes, which may advantageously be substituted polysiloxanes, preferably of low melting point.

The term “silicone wax” is intended to mean an oil comprising at least one silicon atom, and in particular comprising Si—O groups.

Among the commercial silicone waxes of this type, mention may be made in particular of those sold under the names Abilwax 9800, 9801 or 9810 (Goldschmidt), KF910 and KF7002 (Shin-Etsu), or 176-1118-3 and 176-11481 (General Electric).

The silicone waxes that may be used may also be alkyl or alkoxy dimethicones, and also (C₂₀-C₆₀)alkyl dimethicones, in particular (C₃₀-C₄₅)alkyl dimethicones, such as the silicone wax sold under the name SF-1642 by the company GE-Bayer Silicones or C₃₀₋₄₅ alkyl dimethylsilyl polypropylsilsesquioxane under the name SW-8005® C30 Resin Wax sold by the company Dow Corning.

In the context of the present invention, mention may be made, by way of particularly advantageous wax, of beeswax, for example the product sold under the name White Beeswax SP-453P by the company Strahl & Pitsch, or a paraffin wax.

Semi-Crystalline Polymer

The cosmetic composition according to the invention may comprise at least one semi-crystalline polymer. Preferably, the semi-crystalline polymer has an organic structure, and a melting point of greater than or equal to 30° C.

For the purposes of the invention, the term “semi-crystalline polymer” is intended to mean polymers comprising a crystallizable portion and an amorphous portion and having a first-order reversible change of phase temperature, in particular of melting point (solid-liquid transition). The crystallizable part is either a side chain (or pendent chain) or a block in the backbone.

When the crystallizable part of the semi-crystalline polymer is a block of the polymer backbone, this crystallizable block has a chemical nature different from that of the amorphous blocks; in this case, the semi-crystalline polymer is a block copolymer, for example of the diblock, triblock or multiblock type. When the crystallizable part is a chain that is pendent on the backbone, the semi-crystalline polymer may be a homopolymer or a copolymer.

The melting point of the semi-crystalline polymer is preferably less than 120° C.

The melting point of the semi-crystalline polymer is preferably greater than or equal to 40° C. and less than 85° C.

The semi-crystalline polymer(s) according to the invention are solid at ambient temperature (25° C.) and atmospheric pressure (760 mmHg), with a melting point of greater than or equal to 30° C. The melting point values correspond to the melting point measured using a differential scanning calorimeter (DSC), such as the calorimeter sold under the name DSC 30 by the company Mettler, with a temperature rise of 5° C. or 10° C. per minute. The melting point under consideration is the point corresponding to the temperature of the most endothermic peak in the thermogram.

Besides the crystallizable chains or blocks, the blocks of the polymers are amorphous. For the purposes of the invention, the term “crystallizable chain or block” is intended to mean a chain or block which, if it were alone, would change from the amorphous state to the crystalline state reversibly, depending on whether the temperature is above or below the melting point. For the purposes of the invention, a “chain” is a group of atoms, which are pendent or lateral relative to the polymer backbone. A “block” is a group of atoms belonging to the backbone, this group constituting one of the repeating units of the polymer.

The crystallizable blocks or chains of the semi-crystalline polymers may represent at least 30% and better still at least 40% of the total weight of each polymer. The semi-crystalline polymers containing crystallizable side chains are homopolymers or copolymers. The semi-crystalline polymers of the invention containing crystallizable blocks are block or multiblock copolymers. They may be obtained via polymerization of a monomer containing reactive double bonds (or ethylenic bonds) or via polycondensation. When the polymers of the invention are polymers containing crystallizable side chains, these side chains are advantageously in random or statistical form.

The semi-crystalline polymers of the invention may be of synthetic origin.

In particular, the semi-crystalline polymer may be chosen from:
homopolymers and copolymers comprising units resulting from the polymerization of one or more monomers bearing crystallizable hydrophobic side chain(s),
polymers bearing in the backbone at least one crystallizable block,
polycondensates of aliphatic or aromatic or aliphatic/aromatic polyester type,
copolymers of ethylene and propylene prepared via metallocene catalysis, and
acrylate/silicone copolymers.

The semi-crystalline polymers that may be used in the invention may be chosen in particular from:
block copolymers of polyolefins of controlled crystallization, whose monomers are described in EP 0 951 897,
polycondensates, in particular of aliphatic or aromatic or aliphatic/aromatic polyester type,
copolymers of ethylene and propylene prepared via metallocene catalysis,
homopolymers or copolymers bearing at least one crystallizable side chain and homopolymers or copolymers bearing in the backbone at least one crystallizable block, such as those described in document U.S. Pat. No. 5,156,911, such as the (C₁₀-C₃₀)alkyl polyacrylates corresponding to the Intelimer® products from the company Landec described in the brochure Intelimer® Polymers, Landec IP22 (Rev. 4-97), for example the product Intelimer® IPA 13-1 from the company Landec, which is a polystearyl acrylate with a molecular weight of about 145 000 and a melting point of 49° C.,
homopolymers or copolymers bearing at least one crystallizable side chain, in particular containing fluoro group(s), as described in document WO 01/19333,
acrylate/silicone copolymers, such as copolymers of acrylic acid and of stearyl acrylate bearing polydimethylsiloxane grafts, copolymers of stearyl methacrylate bearing polydimethylsiloxane grafts, copolymers of acrylic acid and of stearyl methacrylate bearing polydimethylsiloxane grafts, copolymers of methyl methacrylate, butyl methacrylate, 2-ethylhexyl acrylate and stearyl methacrylate bearing polydimethylsiloxane grafts. Mention may be made in particular of the copolymers sold by the company Shin-Etsu under the names KP-561 (CTFA name: acrylates/dimethicone), KP-541 (CTFA name: acrylates/dimethicone and isopropyl alcohol), KP-545 (CTFA name: acrylates/dimethicone and cyclopentasiloxane),
and mixtures thereof.

In the context of the present invention, as particularly advantageous semi-crystalline polymers, mention may be made of poly(C₁₀-C₃₀)alkyl acrylates, for example the product sold under the name Intelimer IPA 13-1 NG by the company Air products and Chemical.

Preferably, in the context of the present invention, the meltable compound(s) is (are) chosen from ethylene-vinyl acetate (EVA), a beeswax, a paraffin wax, a poly(C₁₀-C₃₀)alkyl acrylate, a vinyl acetate/allyl stearate copolymer, and mixtures thereof.

In the context of the present invention, mention may in particular be made of vinyl acetate/allyl stearate copolymers, for example the product sold under the name Mexomere PQ by the company Chimex.

Aqueous Phase

The cosmetic composition of an assembly according to the invention may comprise an aqueous phase, which may form a continuous phase of the composition.

The aqueous phase may comprise water. It may also comprise at least one water-soluble solvent.

In the context of the present invention, the term “water-soluble solvent” denotes a compound that is liquid at ambient temperature and water-miscible.

The water-soluble solvents that may be used in the compositions according to the invention may also be volatile.

Among the water-soluble solvents that may be used in the compositions in accordance with the invention, mention may be made in particular of lower monoalcohols containing from 1 to 5 carbon atoms such as ethanol and isopropanol, and glycols containing from 2 to 8 carbon atoms such as ethylene glycol, propylene glycol, 1,3-butylene glycol and dipropylene glycol.

The aqueous phase, for example composed of water and optionally a water-miscible solvent, is generally present in a composition according to the invention in a content ranging from 30% to 80% by weight, preferably ranging from 40% to 70% by weight, relative to the total weight of the composition.

Volatile Solvent

A cosmetic composition according to the present invention may comprise one or more volatile solvent(s).

In the context of the present invention, the term “volatile solvent” is intended to mean a compound which is liquid at ambient temperature (20° C.) and atmospheric pressure, having a vapour pressure at 20° C. of greater than 0.1 mmHg and preferably of between 0.1 and 300 mmHg, even more preferentially between 0.5 and 200 mmHg.

This volatile solvent may be water, a non-silicone organic solvent, a silicone organic solvent, or mixtures thereof. By way of volatile non-silicone organic solvent, mention may be made of:
C₁-C₄ volatile alkanols, such as ethanol or isopropanol;
C₅-C₇ volatile alkanes, such as n-pentane, hexane, cyclopentane, 2,3-dimethylbutane, 2,2-dimethylbutane, 2-methylpentane or 3-methylpentane;
esters of liquid C₁-C₂₀ acids and of volatile C₁-C₈ alcohols, such as methyl acetate, n-butyl acetate, ethyl acetate, propyl acetate, isopentyl acetate or ethyl 3-ethoxypropionate;
ketones that are liquid at ambient temperature and volatile, such as methyl ethyl ketone, methyl isobutyl ketone, diisobutyl ketone, isophorone, cyclohexanone or acetone;
volatile polyols, such as propylene glycol;
volatile ethers, such as dimethoxymethane, diethoxyethane or diethyl ether;
volatile glycol ethers, such as 2-butoxyethanol, butyl diglycol, diethylene glycol monomethyl ether, propylene glycol n-butyl ether or propylene glycol monomethyl ether acetate;
volatile hydrocarbon-based oils, such as volatile hydrocarbon-based oils having from 8 to 16 carbon atoms, and mixtures thereof, and in particular branched C₈-C₁₈ alkanes such as C₈-C₁₈ isoalkanes (also known as isoparaffins), isododecane or isodecane, and, for example, the oils sold under the trade names Isopar or Permethyl, and mixtures thereof. Mention may also be made of isohexyl or isodecyl neopentanoates;
volatile C₄-C₁₀ perfluoroalkanes, such as dodecafluoropentane, tetradecafluorohexane or decafluoropentane;
volatile perfluorocycloalkyls, such as perfluoromethylcyclopentane, 1,3-perfluorodimethylcyclohexane and perfluorodecaline, sold respectively under the names Flutec PC10, Flutec PC30 and Flutec PC60 by the company F2 Chemicals, and also perfluorodimethylcyclobutane and perfluoromorpholine;
the volatile fluoroalkyl or heterofluoroalkyl compounds corresponding to the following formula:

CH₃—(CH₂)ₙ—[Z]ₜ—X—CF₃

in which t is 0 or 1; n is 0, 1, 2 or 3; X is a linear or branched divalent perfluoroalkyl radical containing from 2 to 5 carbon atoms, and Z represents O, S or NR, R being a hydrogen, a —(CH₂)ₙ—CH₃ or —(CF₂)ₘ—CF₃ radical, m being equal to 2, 3, 4 or 5.

Among the volatile fluoroalkyl or heterofluoroalkyl compounds, mention may in particular be made of the methoxynonafluorobutane sold under the name MSX 4518® and HFE-7100® by the company 3M and the ethoxynonafluorobutane sold under the name HFE-7200® by the company 3M.

Preferably, the solvent is chosen in such a way that its boiling point is below 200° C.

According to one particular embodiment, the non-silicone organic solvent is chosen from ethanol, isopropanol, acetone and isododecane.

By way of volatile silicone solvent, mention may be made of silicone compounds with a low viscosity, chosen from linear or cyclic silicones having from 2 to 7 silicon atoms, these silicones optionally comprising alkyl or alkoxy groups having from 1 to 10 carbon atoms, for example octamethylcyclotetrasiloxane, decamethylcyclopentasiloxane, dodecamethylcyclohexasiloxane, heptamethylhexyltrisiloxane, heptamethylethyltrisiloxane, heptamethyloctyltrisiloxane, octamethyltrisiloxane, decamethyltetrasiloxane, and mixtures thereof. According to one particular embodiment, the silicone compound is chosen from cyclopentadimethylsiloxane and dodecamethylcyclohexasiloxane.

According to one particular embodiment, the volatile silicone solvent has a viscosity of less than 50 centistokes.

The volatile silicone is preferably cyclic and chosen from decamethylcyclopentasiloxane, octamethyltrisiloxane and decamethyltetrasiloxane.

By way of example, mention may be made of the decamethylcyclopentasiloxane sold under the name DC-245 by the company Dow Corning, the octamethyltrisiloxane sold under the name DC-200 Fluid 1 cst by the company Dow Corning, and the decamethyltetrasiloxane sold under the name DC-200 Fluid 1.5 est by the company Dow Corning.

This cyclic volatile silicone generally has a low viscosity, for example a viscosity of less than 5 cSt at 25° C.

Preferably, the volatile silicone is cyclic and is the decamethylcyclopentasiloxane sold under the name DC-245 by the company Dow Corning.

Preferably, the cosmetic composition comprises less than 20% of volatile solvent(s), preferably less than 10% of volatile solvent(s), and even more preferentially the cosmetic composition is free of volatile solvent(s).

In this first embodiment variant, the composition solidifies when its temperature drops back down to a value less than or equal to its melting point.

The cavity or cavities of the mould can be formed by bringing two jaws together, the jaws remaining closed until the composition has solidified.

The composition may be already present in the mould before the introduction of the fibres, in particular in the case of a disposable mould which is put in place on the jaws at the time of use. In this case, the mould with the composition may optionally be packaged in an individual packaging.

The composition may be brought to a temperature greater than or equal to its melting point outside the mould, in particular in the case where it is injected into the mould, for example via one or more injection channels.

When the composition contains a volatile solvent, and is packaged in the mould, it is preferably contained in a sealed packaging.

The cavity or cavities of the mould can be formed by bringing two jaws together, one or both jaws comprising a heating element. The heating element may be a resistive element, a radiative element, or a device which generates waves, such as microwaves or ultrasound.

The heating element makes it possible to heat the composition in order to bring it to a temperature greater than or equal to its melting point.

The composition may also comprise a material which absorbs certain frequencies and the device may comprise an electromagnetic source e mitting at such frequencies, in the microwave, infrared or visible light range for example.

It is possible to use a source of visible light as heating element and a composition comprising a dye or pigment, the absorption spectrum of which facilitates the absorption of the light. It is likewise possible to use microwaves as heating element with a composition which absorbs these energies, owing, for example, to the presence of water, or to use IR radiation as heating element with a composition which absorbs in the IR range.

The device may also be brought into contact with or exposed to an external energy or heat source. For example, the composition is contained in a mould which is placed in an oven or heated in a water bath before use.

The device may comprise a system for admission, into the mould, of the composition which can be heated by a heating element outside the mould before it reaches the mould cavity or cavities, at a temperature greater than or equal to its melting point.

The device may comprise a system of forced cooling, for example by the Peltier effect, for accelerating the cooling of the composition and the demoulding.

In particular, the device may be arranged so as to lower the temperature of the composition once moulded on the keratin fibres, in particular human keratin fibres, to a temperature below ambient temperature, in particular to a temperature of between 1 and 15° C. prior to demoulding. The Peltier-effect element may be integrated into the jaws.

B. Fluid Composition Containing Particles in the Dispersed State

According to a second embodiment variant, the cosmetic composition of the process or of the assembly according to the invention is fluid at ambient temperature and contains at least particles in the dispersed state in a liquid medium, said composition being solidifiable via the aggregation of said particles.

Thus, the present invention relates to a process for cosmetic treatment of one or more keratin fibres, in particular of one or more eyelashes or eyebrows, comprising at least the step consisting in moulding a fluid cosmetic composition, containing at least particles in the dispersed state in a liquid medium, said composition being solidifiable via the aggregation of said particles, onto at least one part of said fibres, by means of one or more cavities (5), of a mould (2), into which said fibres are at least partially introduced.

According to another of its aspects, the invention relates to a cosmetic assembly, of use for the treatment of keratin fibres, in particular the eyelashes and the eyebrows, comprising:
a fluid cosmetic composition containing at least particles in the dispersed state in a liquid medium, said composition being solidifiable via the aggregation of said particles; and
a device (1) comprising a mould (2), the mould (2) comprising at least one cavity (5), and preferably several cavities (5), into which or each of which at least one of said fibres can be at least partially introduced for the purposes of moulding said composition onto at least one part of said fibre(s) present in the cavity or cavities (5).

According to yet another of its aspects, the present invention relates to the use of a fluid cosmetic composition containing at least particles in the dispersed state in a liquid medium, said composition being solidifiable via the aggregation of said particles, for application thereof as a moulding material in at least one cavity of a mould, for the purposes of depositing on keratin fibres, in particular eyelashes and eyebrows, via the application of said mould to said fibres.

According to this embodiment variant, a composition according to the invention may advantageously have a viscosity of less than 100 Pa·s, preferably between 0.01 Pa·s and 50 Pa·s, preferably between 0.1 Pa·s and 50 Pa·s, and more preferably between 5 Pa·s to 50 Pa·s, at ambient temperature and pressure, the viscosity being in particular measured using a Rheomat RM100®.

In the context of the present invention, it is the reorganization of the particles dispersed in the liquid medium which produces the setting of the particles.

To do this, a composition may comprise from 10% to 55% by weight and preferably from 12% to 50% by weight of particles in the dispersed state, relative to the total weight of the composition.

These particles in the solid state are present, in the composition according to the invention, in a state dispersed in the associated liquid medium. Thus, a composition according to the invention may be in the form of a colloid or else of a suspension.

As previously mentioned, the cosmetic composition is solidifiable via the aggregation of the particles in the dispersed state.

The particles aggregate under the action of an increase in the temperature and/or by evaporation of the liquid medium. Therefore, the composition goes from a fluid state to a thicker state, and better to a solid state.

For the purposes of the invention, the description “aggregation” is intended to characterize the fact that a sufficiently cohesive material or deposit is obtained, and that such material or deposit may be isolated.

A macroscopically continuous deposit, and preferably cohesive, is thus obtained. More preferably, a reportable deposit which can be individually manipulated is obtained. For example, such deposit may be obtained when the deposit is made by pouring onto a non-stick surface such as a Teflon or silicone surface.

Therefore, the present invention is different from a composition which solidifies by aggregation but which does not become cohesive. For example, a composition according to the present invention is different from a composition consisting in water and pigments which by drying will aggregates. Indeed, the obtained deposit will be powdery but not cohesive.

Thus, according to a preferred embodiment, the fluid cosmetic composition of the cosmetic assembly according to the present invention provides, after its solidification via the aggregation of the particles, a cohesive material or deposit preferably insensitive to water. In particular, such cohesive material or deposit exhibits a water uptake of less than or equal to 10%, preferably less than or equal to 5%, and more preferably less than or equal to 3%.

For the purposes of the invention, the description “water uptake” is understood to mean the percentage of water absorbed by the material or the deposit after 60 minutes of immersion in water at 25° C. (ambient temperature).

The water uptake is measured for a layer of composition 300 μm thick (before drying), laid down with the aid of a 300μ applicator on a sheet of glass equipped with a layer of Teflon-coated tape and then dried at 30° C. for 24 hours on a thermostated plate. Three pieces measuring approximately 1 cm² are cut from the dry film and then weighed (mass measurement M1), and then are immersed in water for 60 minutes; following immersion, the piece of film is wiped to removed the excess surface water and then weighed again (mass measurement M2). The difference M2−M1 corresponds to the amount of water absorbed by the film. The water uptake is equal to [(M2−M1)/M1]×100 and is expressed as a percentage by weight of water relative to the weight of the material or the deposit.

Thus, according to one embodiment, the aggregation of the particles dispersed in the liquid medium can be induced by at least partial, or even total, evaporation of said liquid medium.

Preferably, according to this embodiment, the evaporation of said liquid medium is carried out at a temperature of between 45° C. and 90° C., preferably between 50° C. and 80° C.

The evaporation of the liquid medium can also be carried out at ambient temperature, by being in particular stimulated by a forced ventilation, using for example a hairdryer.

Thus, such a composition according to the invention lends itself to thickening via the partial or total evaporation of the liquid ingredients, most commonly represented predominantly by water.

As mentioned above, a composition according to the invention, subject to this phenomenon of evaporation of the liquid ingredients forming its liquid medium, has the advantage of solidifying to form, in the end, a totally cohesive solid material.

This phenomenon is to be distinguished from a phase transition in the true sense, during the passing from one state of the material to another, such as crystallization.

As previously seen, the cosmetic composition comprises particles in the dispersed state and a liquid medium.

Said liquid medium may be, for example, chosen from water, ethanol or mixtures thereof.

The particles in the dispersed state may be chosen from a wax, a film-forming polymer, and mixtures thereof, and in particular may be chosen from a mixture of a film forming polymer and a wax.

In particular, it may be a question of wax emulsions, a microdispersion of wax in water, a dispersion of wax in an anhydrous solvent or else a film-forming polymer dispersed in water.

Preferably, the cosmetic composition comprises film-forming polymer(s) under the form of particles in the dispersed state and a liquid medium.

Wax

It may in particular be a wax as defined above.

According to one embodiment of the invention, a composition according to the invention advantageously comprises from 0% to 35% by weight, in particular from 5% to 30% by weight of wax(es), or even from 10% to 25% by weight of wax(es), relative to the total weight of the composition.

In another embodiment, a composition according to the invention comprises an amount of wax of less than 8%, or even less than 3%, and even better still less than 1% by weight, relative to the total weight of the composition.

In the context of the present invention, mention may be made, by way of particularly advantageous wax, of carnauba wax, advantageously in the form of a microdispersion, for example the one sold under the name Mexoryl SAP by the company Chimex.

Film Forming Polymer

A composition according to the invention may preferably comprise at least an aqueous dispersion of film-forming polymer particles and optionally at least one additional film-forming polymer (not present in the form of an aqueous dispersion of particles, such as a water-soluble film-forming polymer).

In the present application, the term “film-forming polymer” is intended to mean a polymer that is capable, by itself or in the presence of an auxiliary film-forming agent, of forming a macroscopically continuous deposit, and preferably a cohesive deposit, and even better still a deposit of which the cohesion and mechanical properties are such that said deposit can be isolated and manipulated individually, for example when said deposit is prepared by pouring onto a non-stick surface such as a Teflon-coated or silicone-coated surface.

A composition according to the invention preferably comprises a total solids content of film-forming polymer(s) of greater than or equal to 5% by weight, preferably greater than or equal to 10% by weight and better still greater than or equal to 12% by weight, relative to the total weight of the composition.

A composition according to the invention preferably comprises a total solids content of film-forming polymer(s) ranging from 10% to 55% by weight, in particular from 12% to 50% by weight, relative to the total weight of the composition.

In particular, a composition according to the invention preferably comprises at least an aqueous dispersion of particles formed from one or more film-forming polymers.

It may also comprise at least one water-soluble film-forming polymer.

Thus, a composition according to the invention may comprise at least one additional film-forming polymer, different from the film-forming polymer particles present in aqueous dispersion form.

The content of this (these) “water-soluble” additional film-forming polymer(s) is preferably less than or equal to 10% by weight, even more preferentially less than or equal to 5% by weight and better still less than or equal to 2% by weight, relative to the total weight of the composition.

Film-Forming Polymer(s) in Aqueous Dispersion

Such a film-forming polymer present in said preparation of the composition in the form of particles in aqueous dispersion is generally known as a (pseudo)latex, i.e. a latex or pseudolatex. Techniques for preparing these dispersions are well known to those skilled in the art.

A dispersion that is suitable for use in the invention may comprise one or more types of particle, these particles possibly varying as regards their size, their structure and/or their chemical nature.

A composition according to the invention may comprise a total solids content of film-forming polymer particles in aqueous dispersion form of greater than or equal to 10% by weight, relative to the total weight of the composition.

Advantageously, a composition according to the invention comprises a total solids content of film-forming polymer particles in aqueous dispersion form of greater than or equal to 12% by weight, relative to the total weight of the composition.

A composition according to the invention preferably comprises a total solids content of film-forming polymer particles ranging from 10% to 55% by weight, better still from 12% to 50% by weight, relative to the total weight of the composition.

The total content of film-forming polymer particles present in aqueous dispersion form is preferably greater than or equal to 30% by weight and preferentially greater than or equal to 40% by weight, relative to the total weight of the particles.

These particles may be of anionic, cationic or neutral nature and may constitute a mixture of particles of different nature.

Among the film-forming polymers that may be used in the composition of the present invention, mention may be made of synthetic polymers, of free-radical type or of polycondensate type, and polymers of natural origin, and mixtures thereof. In general, these polymers may be random polymers, block copolymers of A-B type, multiblock copolymers A-B-A or else of ABCD type, etc., or even grafted polymers.

Free-radical film-forming polymer:

The term “free-radical polymer” is intended to mean a polymer obtained by polymerization of unsaturated and in particular ethylenically unsaturated monomers, each monomer being capable of homopolymerizing (unlike polycondensates).

The film-forming polymers of free-radical type may in particular be acrylic and/or vinyl homopolymers or copolymers.

The vinyl film-forming polymers may result from the polymerization of ethylenically unsaturated monomers containing at least one acid group and/or esters of these acid monomers and/or amides of these acid monomers.

Ethylenically unsaturated monomers bearing at least one acid group or monomer bearing an acid group that may be used include α,β-ethylenic unsaturated carboxylic acids such as acrylic acid, methacrylic acid, crotonic acid, maleic acid or itaconic acid. (Meth)acrylic acid and crotonic acid are in particular used, and more particularly (meth)acrylic acid.

The esters of acid monomers are advantageously chosen from (meth)acrylic acid esters (also known as (meth)acrylates), especially (meth)acrylates of an alkyl, in particular of a C₁-C₂₀ and more particularly C₁-C₈ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl (meth)acrylates that may be mentioned are methyl methacrylate, ethyl methacrylate, butyl methacrylate, isobutyl methacrylate, 2-ethylhexyl methacrylate and lauryl methacrylate.

Among the hydroxyalkyl (meth)acrylates that may be mentioned are hydroxyethyl acrylate, 2-hydroxypropyl acrylate, hydroxyethyl methacrylate and 2-hydroxypropyl methacrylate.

Among the aryl (meth)acrylates that may be mentioned are benzyl acrylate and phenyl acrylate.

The (meth)acrylic acid esters are in particular alkyl (meth)acrylates.

According to the present invention, the alkyl group of the esters may be either fluorinated or perfluorinated, i.e. some or all of the hydrogen atoms of the alkyl group are substituted with fluorine atoms.

Examples of amides of acid monomers that may be mentioned are (meth)acrylamides, and in particular N-alkyl(meth)acrylamides, in particular of a C₂-C₁₂ alkyl. Among the N-alkyl(meth)acrylamides that may be mentioned are N-ethylacrylamide, N-t-butylacrylamide and N-t-octylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned previously.

Examples of vinyl esters that may be mentioned are vinyl acetate, vinyl neodecanoate, vinyl pivalate, vinyl benzoate and vinyl t-butylbenzoate.

Styrene monomers that may be mentioned include styrene and α-methylstyrene.

The list of monomers given is not limiting, and it is possible to use any monomer known to those skilled in the art included in the categories of acrylic and vinyl monomers (including monomers modified with a silicone chain).

Vinyl polymers that may also be used include silicone acrylic polymers.

Mention may also be made of polymers resulting from free-radical polymerization of one or more free-radical monomers inside and/or partially at the surface of pre-existing particles of at least one polymer chosen from the group consisting of polyurethanes, polyureas, polyesters, polyesteramides and/or alkyds. These polymers are generally referred to as “hybrid polymers”.

Polycondensate:

As film-forming polymer of polycondensate type, mention may be made of anionic, cationic, nonionic or amphoteric polyurethanes, acrylic polyurethanes, polyvinylpyrrolidone-polyurethanes, polyester-polyurethanes, polyether-polyurethanes, polyureas, polyurea/polyurethanes and silicone polyurethanes, and mixtures thereof.

The film-forming polyurethane may be, for example, an aliphatic, cycloaliphatic or aromatic polyurethane, polyurea/urethane or polyurea copolymer comprising, alone or as a mixture, at least one block chosen from:
a block of aliphatic and/or cycloaliphatic and/or aromatic polyester origin, and/or
a branched or unbranched silicone block, for example polydimethylsiloxane or polymethylphenylsiloxane, and/or
a block comprising fluoro groups.

The film-forming polyurethanes as defined in the invention may also be obtained from branched or unbranched polyesters or from alkyds comprising mobile hydrogens, which are modified by reaction with a diisocyanate and a difunctional organic compound (for example dihydro, diamino or hydroxyamino), also comprising either a carboxylic acid or carboxylate group, or a sulfonic acid or sulfonate group, or alternatively a neutralizable tertiary amine group or a quaternary ammonium group.

Among the film-forming polycondensates, mention may also be made of polyesters, polyesteramides, fatty-chain polyesters, polyamides and epoxyester resins.

The polyesters may be obtained, in a known manner, by polycondensation of dicarboxylic acids with polyols, in particular diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norboranedicarboxylic acid, diglycolic acid, thiodipropionic acid, 2,5-naphthalenedicarboxylic acid, and 2,6-naphthalenedicarboxylic acid. These dicarboxylic acid monomers may be used alone or as a combination of at least two dicarboxylic acid monomers. Among these monomers, the ones chosen in particular are phthalic acid, isophthalic acid and terephthalic acid.

The diol may be chosen from aliphatic, alicyclic and aromatic diols. The diol used is chosen in particular from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

Polymer of natural origin:

Use may be made in the present invention of optionally modified polymers of natural origin, such as shellac resin, sandarac gum, dammar resins, elemi gums, copal resins, water-insoluble cellulose-based polymers such as nitrocellulose, modified cellulose esters in particular including carboxyalkyl cellulose esters such as those described in patent application US 2003/185 774, and mixtures thereof.

According to a particular embodiment of the invention, said at least one film-forming polymer in the dispersed state is chosen from acrylic polymer dispersions, polyurethane dispersions, sulfopolyester dispersions, vinyl dispersions, polyvinyl acetate dispersions, vinylpyrrolidone, dimethylaminopropylmethacrylamide and lauryldimethylpropylmethaerylamidoammonium chloride terpolymer dispersions, polyurethane/polyacrylic hybrid polymer dispersions and dispersions of particles of core-shell type, and mixtures thereof.

Various types of aqueous dispersion, in particular commercial dispersions, which are suited to the preparation of the composition in accordance with the present invention are detailed below.

1/Thus, according to one preferred embodiment of the invention, the aqueous dispersion of polymer particles is an aqueous dispersion of acrylic polymer.

The acrylic polymer can be a styrene/acrylate copolymer and in particular a polymer chosen from copolymers resulting from the polymerization of at least one styrene monomer and at least one C₁-C₁₈ alkyl (meth)acrylate monomer.

As styrene monomer that may be used in the invention, examples that may be mentioned include styrene and α-methylstyrene, and in particular styrene.

The C₁-C₁₈ alkyl (meth)acrylate monomer is in particular a C₁-C₁₂ alkyl (meth)acrylate and more particularly a C₁-C₁₀ alkyl (meth)acrylate. The C₁-C₁₈ alkyl (meth)acrylate monomer may be chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate.

As acrylic polymer in aqueous dispersion, use may be made according to the invention of the styrene/acrylate copolymer sold under the name Joncryl SCX-8211® by the company BASF or Syntran 5760cg by the company Interpolymer, the acrylic polymer sold under the reference Acronal® DS-6250 by the company BASF, or the acrylic copolymer Joncryl® 95 by the company BASF.

2/According to one embodiment of the invention, the aqueous dispersion of polymer particles is an aqueous dispersion of polyester-polyurethane and/or polyether-polyurethane particles, in particular in anionic form.

The anionic nature of the polyester-polyurethanes and of the polyether-polyurethanes used according to the invention is due to the presence in their constituent units of groups bearing a carboxylic acid or sulfonic acid function.

The polyester-polyurethane or polyether-polyurethane particles used according to the invention are generally sold in aqueous dispersion form.

The particle content of said dispersions currently available on the market ranges from approximately 20% to approximately 60% by weight relative to the total weight of the dispersion.

Among the anionic polyester-polyurethane dispersions that may be used in the compositions according to the invention, mention may be made in particular of the product sold under the name Avalure UR 405® by the company Noveon or Baycusan C1004 by the company Bayer Material Science.

Among the anionic polyether-polyurethane particle dispersions that may be used according to the invention, mention may be made in particular of the products sold under the name Avalure UR 450® by the company Noveon and under the name Neorez R 970® by the company DSM.

According to a particular embodiment of the invention, use may be made of a mixture of commercial dispersions consisting of anionic polyester-polyurethane particles as defined above and of anionic polyether-polyurethane particles also defined above.

For example, use may be made of a mixture consisting of the dispersion sold under the name Sancure 861® or a mixture of the product sold under the name Avalure UR 405® and of the product sold under the name Avalure UR 450®, these dispersions being sold by the company Noveon.

3/According to another particular embodiment of the invention, the aqueous dispersion used comprises a mixture of at least two film-forming polymers in the form of particles that differ by their respective glass transition temperatures (Tg).

In particular, according to one embodiment of the invention, the composition in accordance with the invention may comprise at least a first film-forming polymer in the dispersed state and at least a second film-forming polymer in the dispersed state, said first and second polymers having different Tg values and, preferably, the Tg of the first polymer (Tg1) is higher than the Tg of the second polymer (Tg2). In particular, the difference between the Tg1 and Tg2 values is, as an absolute value, at east 10° C. and preferably at least 20° C.

More precisely, it comprises in an acceptable aqueous medium:

a) particles dispersed in the aqueous medium of a first film-forming polymer having at least one glass transition temperature Tg1 greater than or equal to 20° C., and

b) particles dispersed in the aqueous medium of a second film-forming polymer having at least one glass transition temperature Tg2 less than or equal to 70° C.

This dispersion generally results from a mixing of two aqueous dispersions of film-forming polymer.

The first film-forming polymer has at least one glass transition temperature Tg1 greater than or equal to 20° C., in particular ranging from 20° C. to 150° C. and advantageously greater than or equal to 40° C., especially ranging from 40° C. to 150° C. and in particular greater than or equal to 50° C., especially ranging from 50° C. to 150° C.

The second film-forming polymer has at least one glass transition temperature Tg2 less than or equal to 70° C., especially ranging from −120° C. to 70° C., and in particular less than 50° C., especially ranging from −60° C. to +50° C. and more particularly ranging from −30° C. to 30° C.

The measurement of the glass transition temperature (Tg) of a polymer is performed by DMTA (dynamic and mechanical temperature analysis) as described below.

To measure the glass transition temperature (Tg) of a polymer, viscoelasticity tests are performed with a “Polymer Laboratories” DMTA machine, on a sample of film. This film is prepared by pouring the aqueous dispersion of film-forming polymer into a Teflon-coated matrix and then dried at 120° C. for 24 hours. A film is then obtained, from which specimens are cut out (for example using a punch). These specimens are typically about 150 μM thick, from 5 to 10 mm wide and have a useful length of about 10 to 15 mm. A tensile stress is imposed on this sample. The sample undergoes a static force of 0.01 N on which is superimposed a sinusoidal displacement of ±8 μm at a frequency of 1 Hz. The test is thus performed in the linear range, at low levels of deformation. This tensile stress is performed on the sample at temperatures ranging from −150° C. to +200° C., with a temperature variation of 3° C. per minute.

The complex modulus E*=E′+iE″ of the polymer tested is thus measured as a function of the temperature.

From these measurements, the dynamic moduli E′ and E″ and the damping power: tgδ=E″/E′ are deduced.

The curve of the Tgδ values is then plotted as a function of the temperature; this curve presents at least one peak. The glass transition temperature Tg of the polymer corresponds to the temperature at the top of this peak.

When the curve presents at least two peaks (in this case, the polymer has at least two Tg values), the value taken as the Tg of the polymer tested is the temperature for which the curve presents a peak of the largest amplitude (i.e. corresponding to the largest Tg6 value; in this case, only the “major” Tg is considered as the Tg value of the polymer tested).

In the present invention, the transition temperature Tg1 corresponds to the “major” Tg (in the predefined sense) of the first film-forming polymer when the latter has at least two Tg values; the glass transition temperature Tg2 corresponds to the “major” Tg of the second film-forming polymer when the latter has at least two Tg values.

The first film-forming polymer and the second film-forming polymer may be chosen, independently of each other, from free-radical polymers, polycondensates and polymers of natural origin as defined previously having the glass transition temperature characteristics defined previously.

As first film-forming polymer in aqueous dispersion, use may be made of the aqueous polymer dispersions sold under the names Neorez R-989® by the company DSM, Joncryl 95 and Joncryl® 8211 by the company BASF.

As second film-forming polymer in aqueous dispersion, use may be made, for example, of the aqueous polymer dispersions sold under the names Avalure® UR-405, Avalure® UR-460 by the company Noveon or Acrilem IC89RT® by the company ICAP, and Neocryl A-45 by the company DSM.

The film-forming polymer of the aqueous dispersion Avalure® UR-460 is a polyurethane obtained by polycondensation of polytetramethylene oxide, tetramethyixylylene diisocyanate, isophorone diisocyanate and dimethylolpropionic acid.

According to a most particularly preferred embodiment of the invention, use is made, as first and second film-forming polymers in aqueous dispersion, of the combination of styrene/acrylate polymer dispersion such as the dispersion sold under the reference Joncryl 8211 n by BASF and of acrylic polymer dispersion such as the dispersion sold under the reference Neocryl A-45® by DSM.

According to another preferred embodiment, use is made, as first film-forming polymer in aqueous dispersion, of an acrylic polymer dispersion such as the dispersion sold under the reference Joncryl 95® by BASF and, as second film-forming polymer, of a dispersion of anionic polyurethane polymer sold under the reference Avalure UR405® by DSM.

As aqueous dispersions of film-forming polymer, use may be made of:
the acrylic dispersions sold under the names Acronal DS-6250® by the company BASF, Neocryl A-45®, Neocryl XK-90®, Neocryl A-1070®, Neocryl A-1090®, Neocryl BT-62®, Neocryl A-1079® and Neocryl A-523® by the company DSM, Joncryl 95® and Joncryl 8211® by the company BASF, Daitosol 5000 AD® or Daitosol 5000 SJ by the company Daito Kasey Kogyo; Syntran 5760 CG by the company Interpolymer,
the aqueous polyurethane dispersions sold under the names Neorez R-981® and Neorez R-974® by the company DSM, Avalure UR-405®, Avalure UR-410®, Avalure UR-425®, Avalure UR-450®, Sancure 875®, Avalure UR 445® and Avalure UR 450® by the company Noveon, Impranil 85® by the company Bayer, and Baycusan C1004® by the company Bayer Material Science,
the sulfopolyesters sold under the brand name Eastman AQ® by the company Eastman Chemical Products,
vinyl dispersions such as Mexomer PAM, aqueous dispersions of polyvinyl acetate such as Vinybran® from the company Nisshin Chemical or the products sold by the company Union Carbide, aqueous dispersions of vinylpyrrolidone, dimethylaminopropylmethacrylamide and lauryldimethylpropylmethacrylamidoammonium chloride terpolymer such as Styleze W® from 1SP,
aqueous dispersions of polyurethane/polyacrylic hybrid polymer such as the products sold under the references Hybridur® by the company Air Products or Duromer® from National Starch,
dispersions of particles of core-shell type such as the products sold by the company Arkema under the reference Kynar® (core:fluorinated-shell:acrylic) or alternatively those described in U.S. Pat. No. 5,188,899 (core:silica-shell:silicone) and mixtures thereof.

According to a preferred embodiment, a composition in accordance with the invention comprises an aqueous dispersion of particles chosen from aqueous dispersions of acrylic film-forming polymer(s) and derivatives, in particular of styrene-acrylic and derivatives, and aqueous dispersions of polyurethane polymer(s), in particular of polyester-polyurethane, and derivatives thereof, and mixtures thereof.

In the context of the present invention, as particularly advantageous film-forming polymer, mention may be made of latexes, for example the product sold under the name Syntran 5760 CG by the company Interpolymer.

According to one particular embodiment, the total content of wax(es) and the total content of film-forming polymer particles are such that the weight ratio of the wax(es) to the film-forming polymer particles is greater than or equal to ½ and better still ⅔.

Preferably, this ratio is inclusively between ½ and 2 and even more preferentially between ⅔ and 3/2.

According to one particular embodiment, the total content of wax(es) and the total content of film-forming polymer particles, which are both preferentially present in the form of particles in aqueous dispersion, with the film-forming polymer(s) chosen from aqueous dispersions of acrylic film-forming polymer(s) and derivatives, in particular styrene-acrylic and derivatives, and aqueous dispersions of polyester-polyurethane hybrid polymer(s), and mixtures thereof, are such that the weight ratio of the wax particles to said film-forming polymer particles is greater than or equal to ½ and better still ⅔.

For example, this ratio is inclusively between ½ and 2 and even more preferentially between ⅔ and 3/2.

Preferably, the particles in the dispersed state may be chosen from a wax, latex, and mixtures thereof.

According to one particular embodiment, a composition according to the invention consists of a particulate dispersion comprising at least one mixture of a microdispersion of carnauba and a latex.

The composition may be packaged in a pressurized container and may comprise a pressurized propellant gas.

During the moulding, the composition may be heated above the vaporization temperature of the liquid medium.

The composition may be ventilated, for example by a stream of air, during the moulding, so as to accelerate the evaporation of the liquid medium.

The opening of the jaws may also contribute to the evaporation of the liquid medium.

A specific absorber of the liquid medium may be introduced during the moulding or prior thereto. The solvent is then at least partly eliminated by absorption. The mould, in particular if it is disposable, may comprise an absorber specific to the liquid medium.

The device may comprise a system which assists the drying of the composition, the system providing energy, in particular in the form of light or heat, and/or comprising an aeration circuit, suction and/or blowing of air.

The cavity or cavities of the mould can be formed by bringing two jaws together, the jaws remaining closed until the composition has solidified.

The device may comprise one or two jaws, it being possible for one or both of the jaws to have a system for admitting drying compounds, such as a calcium salt for example.

The mould may comprise an absorbent material or be covered with absorbent or desiccating material, in particular a ceramic or a porous material, or a chemical active agent which is capable of absorbing, such as a silica gel or at least one compound such as an aerogel, clay, bentonite, calcium or cobalt or copper salts (chlorate, sulfate), lithium halides, potassium carbonate, magnesium sulfate or sodium sulfate, or organic compounds such as sugars.

The keratin fibres, in particular human keratin fibres, may be covered with an absorbent material as described above, before they are introduced into the mould.

C. Fluid Composition Comprising an Activatable Compound

According to a third embodiment variant, a cosmetic composition in a process or an assembly according to the invention is fluid at ambient temperature and contains at least one compound that is activatable by a physical or chemical stimulus, said composition being solidifiable via the conversion of the activated form of said compound into a solid material, by self-reaction and/or interaction with at least one supplementary compound.

Thus, the present invention relates to a process for cosmetic treatment of one or more keratin fibres, in particular of one or more eyelashes or eyebrows, comprising at least the step consisting in moulding a fluid cosmetic composition, containing at least one compound that is activatable by a physical or chemical stimulus, said composition being solidifiable via the conversion of the activated form of said compound into a solid material, by self-reaction and/or interaction with at least one supplementary compound, onto at least one part of said fibres, by means of one or more cavities (5), of a mould (2), into which said fibres are at least partially introduced.

According to another of its aspects, the invention relates to a cosmetic assembly, of use for the treatment of keratin fibres, in particular the eyelashes and the eyebrows, comprising:
a fluid cosmetic composition, containing at least one compound that is activatable by a physical or chemical stimulus, said composition being solidifiable via the conversion of the activated form of said compound into a solid material, by self-reaction and/or interaction with at least one supplementary compound; and
a device (1) comprising a mould (2), the mould (2) comprising at least one cavity (5), and preferably several cavities (5), into which or each of which at least one of said fibres can be at least partially introduced for the purposes of moulding said composition onto at least one part of said fibre(s) present in the cavity or cavities (5).

According to yet another of its aspects, the present invention relates to the use of a fluid cosmetic composition containing at least one compound that is activatable by a physical or chemical stimulus, said composition being solidifiable via the conversion of the activated form of said compound into a solid material, by self-reaction and/or interaction with at least one supplementary compound, for application thereof as a moulding material in at least one cavity of a mould, for the purposes of depositing on keratin fibres, in particular eyelashes and eyebrows, via the application of said mould to said fibres.

Thus, such a composition may advantageously have a viscosity of less than 100 Pa·s, preferably between 0.1 Pa·s and 50 Pa·s, preferably between 1 Pa·s and 50 Pa·s, at ambient temperature and pressure, the viscosity being in particular measured using a Rheomat RM100®.

A composition according to the invention lends itself to solidification via the conversion of the activated form of an activatable compound into a solid material, by self-reaction and/or interaction with at least one supplementary compound.

The crosslinking carried out may be of the AA or AB type.

Thus, the composition is fluid before reaction and becomes solid after reaction.

For the purposes of the invention, an “activatable” compound is a compound or a species which reacts characteristically in response to a physical or chemical stimulus.

In particular, the physical or chemical stimulus may be a variation in light, for example UV or visible light, in pH, in temperature or in moisture content, or else the bringing into contact with at least one reagent which may or may not be different from the supplementary compound.

Thus, in response to such a stimulus, the activated form of the activatable compound is converted into a solid material, then inducing the solidification of the composition containing it.

According to a first embodiment, the composition is solidifiable by self-reaction of the activatable compound.

In this embodiment, the activatable compound reacts with itself or on itself in response to a physical or chemical stimulus.

More particularly, the activatable compound is a photosensitive compound.

Thus, the stimulus may be a variation in light, for example UV or visible light. Such a stimulus may generate crosslinking of the activatable compound.

Thus, the activatable compound is preferably chosen from monomers, oligomers or polymers comprising an acrylate, methacrylate, acrylamide or methacrylamide function and monomers, oligomers or polymers capable of reacting by 2+2 or 2+4 cycloaddition, such as stilbazolium compounds.

These may advantageously be methacrylate compounds sold under the name Power Polish™ Top Coat by the company CND Shellac.

As oligomers or polymers capable of reacting by 2+2 or 2+4 cycloaddition, mention may in particular be made of polyvinylalcohol/acetates grafted with stilbazolium functions.

According to a second embodiment, the composition is solidifiable by interaction of the activatable compound with at least one supplementary compound, the interaction being of covalent or non-covalent type.

In this embodiment, the two activatable and supplementary compounds may react rapidly, and produce, in less than a few minutes, via their conversion, the desired solidification of the composition containing them.

According to a first aspect, the supplementary compound is a compound present in the atmosphere, for example water or oxygen, and the activatable compound is chosen from a siccative oil, a monomer, oligomer or polymer comprising a silane function, a cyanoacrylate, a reactive silicone, and mixtures thereof.

The term “siccative oil” is intended to denote an oil which, when spread as a thin coat and then exposed to the air, transforms into a solid film coating. In particular, the term “siccative oil” is intended to denote, in the context of the present invention, oils, and preferably triglycerides, comprising conjugated double bonds, preferably comprising at least two conjugated double bonds and preferably comprising at least three conjugated double bonds.

The siccative oils in accordance with the invention may be of natural origin. Advantageously, the siccative oil may be chosen from siccative vegetable oils such as linseed oil, Chinese (or Cantonese) wood oil, oiticica oil, vernonia oil, poppy oil, pomegranate oil or marigold oil, esters of these vegetable oils, alkyd resins obtained from these vegetable oils, and mixtures thereof. Alkyd resins are polyesters comprising hydrocarbon-based chains of fatty acids, obtained in particular by the polymerization of polyols and polyacids or the corresponding anhydride thereof, in the presence of fatty acids. These fatty acids are present, in particular in triglyceride form, in the majority of natural oils, such as, in particular, the abovementioned oils.

The siccative oil that is suitable for use in the present invention may be modified by chemical reaction. In particular, it may be refined and/or partially polymerized. In this respect, mention may be made of blown oils and stand oils, and maleinized, epoxidized or boiled oils. The blowing of an oil is characterized in particular by polymerization of said oil with atmospheric oxygen.

As cyanoacrylate that is quite particularly suitable for the invention, mention may be made of 2-octyl-2-cyanoacrylate stabilized with 1% of phosphoric acid, for example Rite Lok CON895, sold by the company Chemence.

Advantageously, the activatable compound is ethyl cyanoacrylate, in particular the product sold under the name Superglue 3 by the company Loctite which may also be mentioned.

The reactive silicones may be chosen from moisture-sensitive reactive silicones.

Preferably, they will be the reactive silicones sold under the name Joint & Fix by the company GEB.

According to a second aspect, the activatable compound and the supplementary compound have, respectively, complementary functional groups.

The term “complementary functional groups” is intended to mean groups which are capable of reacting with one another.

Thus, the activatable compound and the supplementary compound may be, respectively, a nucleophilic compound and an electrophilic compound.

As pairs of nucleophilic compound and electrophilic compound quite particularly suitable for the invention, mention may in particular be made of polyols or polyamines to be reacted with polyanhydrides or polyisocyanates.

Preferably, the nucleophilic compound and the electrophilic compound are a polyamine and a polyanhydride or two distinct reactive silicones.

By way of reactive silicones, mention may quite particularly be made of the Platsil gel 10 (A:B) reactive silicones sold by the company Polytek or else the product Dow Corning 7-FC4210 Elastomer film forming base sold by the company Dow Corning and the product Dow Corning 7-FC4210 Curing Agent sold by the company Dow Corning.

By way of example of polyamine/polyanhydride, use may also be made of diaminopropane to be mixed with Gantrez AN-119 BF from the company Ashland.

The activatable compound and the supplementary compound may also be, respectively, a cationic polymer and an anionic polymer.

As pairs of cationic polymer and anionic polymer quite particularly suitable for the invention, mention may in particular be made of polyacrylates or polymethacrylates or a copolymer (such as UltraHold Strong from the company BASF) having neutralized acid functions (aqueous ammonia, amine, strong base) to be combined with cationic polymers such as JR 400, quaternized polysaccharides (quaternized guar gum of the Jaguar C13S type from the company Rhodia, quaternized HEC), polyDADMAC such as Polyquaternium-7 from the company Nalco.

The activatable compound and the supplementary compound may also be, respectively, a carbonate or alginate compound and a calcium-based compound.

As carbonate compound, mention may in particular be made of sodium, potassium or ammonium carbonate or bicarbonate.

The alginate compounds may be chosen from Protanal PH 6160 from the company FMC Corporation.

By way of calcium-based compounds, mention may be made of calcium chloride and other halides.

Preferably, this involves a solution of sodium alginate such as the product sold under the name Kelcosol by the company ISP and an aqueous solution of calcium chloride.

Catalyst

According to one preferred embodiment, the composition also comprises a catalyst, in particular suitable for accelerating the reaction.

By way of example in the case of the reactive silicones, the catalyst may in particular be chosen from titanium derivatives.

The cavity or cavities of the mould can be formed by bringing two jaws together, the jaws remaining closed until the composition has solidified.

The reactivity of the compound or of the composition can be accelerated by conventional routes, such as providing energy, in particular by light or heat, or a catalyst.

The device may comprise two jaws, at least one of which may comprise a system for admitting at least one reagent. One or both of the jaws may comprise at least two separate systems for admitting reagents, the reagents coming into contact within the mould cavity or cavities.

The device may comprise a chamber for mixing the reagents upstream of the mould cavity or cavities.

The device may comprise a triggering system, such as an illuminator, in particular an IR-, UV- or visible-light illuminator. The mould is then preferably transparent to the wavelengths used to trigger the reaction.

D. Ductile Composition

According to a fourth embodiment variant, the cosmetic composition is ductile and preferably has a Young's modulus of less than or equal to 100 MPa at ambient temperature and pressure.

In this embodiment variant, the composition is combined with a mould that is deformable, under compression or suction, the mould comprising at least one cavity, and preferably several cavities, into which or each of which at least one of said fibres can be at least partially introduced, for the purposes of moulding said composition onto at least one part of said fibre(s) present in the cavity or cavities.

Thus, the present invention relates to a process for cosmetic treatment of one or more keratin fibres, in particular of one or more eyelashes or eyebrows, comprising at least the steps consisting in:
depositing a ductile cosmetic composition preferably having a Young's modulus of less than or equal to 100 MPa, at ambient temperature and pressure, in one or more cavities (5) of a mould (2) that is deformable, under compression or suction, into which cavities said fibres are at least partially introduced; and
subjecting the mould (2) to a compression or to a suction, so as to promote the adhesion of said cosmetic composition deposited in said cavity or cavities, on at least one part of said fibres.

According to another of its aspects, the invention relates to a cosmetic assembly, of use for the treatment of keratin fibres, in particular the eyelashes and the eyebrows, comprising:
a ductile cosmetic composition preferably having a Young's modulus of less than or equal to 100 MPa, at ambient temperature and pressure; and
a device (1) comprising a mould (2) that is deformable, under compression or suction, the mould (2) comprising at least one cavity (5), and preferably several cavities (5), into which or each of which at least one of said fibres can be at least partially introduced for the purposes of moulding said composition onto at least one part of said fibre(s) present in the cavity or cavities (5).

According to yet another of its aspects, the present invention relates to the use of a ductile cosmetic composition preferably having a Young's modulus of less than or equal to 100 MPa, at ambient temperature and pressure, for application thereof as a moulding material in at least one cavity of a mould that is deformable, under compression or suction, for the purposes of depositing on keratin fibres, in particular eyelashes and eyebrows, via the application of said mould to said fibres.

The term “ductile” is intended to describe the capacity of the cosmetic composition to deform plastically without breaking. Breaking occurs when a defect, such as a crack or a cavity, induced by the plastic deformation, becomes critical and propagates. The ductility therefore reflects the ability of the cosmetic composition to resist this propagation.

Thus, the composition under consideration deforms, under compression or under suction, plastically, i.e. it retains at least partly the deformation that it is given by the mould, without breaking.

In particular, the composition, under compression or under suction, deforms and hugs the shape of the keratin fibre(s) introduced into the cavity or cavities of the deformable mould.

Preferably, the cosmetic composition is deformable at a pressure of between 0.02 bar and 100 bar, at ambient temperature.

In addition, the composition is preferably self-healing, i.e. it gives a material which, when it is subjected to a mechanical deformation causing it to produce a crack, said crack heals and, in doing so, the material obtains again all or part of its integrity. The healing operation takes place by itself in a period of time ranging from a few seconds to one hour approximately, by simple contact of the two edges of the crack.

In particular, the composition is preferably pasty, i.e. the consistency of this composition is intermediate between a solid phase and a liquid phase. The viscosity of a pasty composition is advantageously greater than 0.1 Pa·s, and preferably greater than 1 Pa·s, this being at 25° C. with a shear rate of 10 s⁻¹, the viscosity being in particular measured using a Rheomat RM100®.

Thus, the composition under consideration is sufficiently pasty so as not to creep under its own weight under the ambient temperature and pressure conditions. In this case, the composition may advantageously be provided in a shape which helps with the integration of the keratin fibres. For example, striations may be made in the composition. The striations guide the keratin fibres and facilitate the integration within the material.

Preferably, the cosmetic composition has a Young's modulus of less than or equal to 100 MPa at ambient temperature and pressure.

In particular, it may have a Young's modulus of less than or equal to 10 MPa at ambient temperature and pressure.

The Young's modulus characterizes the strength of the composition exposed to a mechanical action. Thus, it characterizes the force to be applied, per unit of surface area, in order to produce a modification of the composition.

Tensile tests are carried out in order to measure the Young's modulus. The film is cut into pieces of rectangular shape, 80 mm long and 15 mm wide.

The tests are performed on a machine sold under the name Lloyd or sold under the name Zwick, under the same temperature and humidity conditions as for the drying, i.e. a temperature of 22+1-2° C. and a relative humidity of 50+/−5%.

The test pieces are drawn at a speed of 20 mm/min and the distance between the jaws is 50+/−1 mm.

In other words, advantageously, the composition according to the present invention has in the dry state an ultimate strain εᵣ of greater than or equal to 15%, preferably greater than or equal to 25%.

The ultimate strain is determined by means of tensile tests performed on a sample of a composition of the invention under the form of a film approximately 200 μm thick.

To perform these tests, the film is cut into dumb-bell-shaped test pieces with a working length of 33±1 mm and a working width of 6 mm. The cross section (S) of the test piece is then defined as: S=width×thickness (cm²); this cross section will be used to calculate the stress.

The tests are performed, for example, on a tensile testing device sold under the name Lloyd® LRSK. The measurements are performed at ambient temperature (20° C.).

The test pieces are drawn at a draw rate of 33 mm/min, corresponding to a rate of 100% elongation per minute.

A draw speed is thus applied and the elongation ΔL of the test piece and the force F required to impose this elongation are simultaneously measured. From these data ΔL and F, the stress σ and strain ε parameters are determined.

A curve of stress σ=(F/S) as a function of the strain ε=([Δ]L/Lₒ)×100 is thus obtained, the test being carried out until the test piece fails, Lₒ being the initial length of the test piece.

The ultimate strain εᵣ is the maximum strain of the sample before the point at which it breaks (as %).

Moreover, the cosmetic composition may exhibit an elastic recovery of less than or equal to 80%, after 40% tensile deformation.

The term “elastic recovery” is intended to mean the degree of return to its initial length of a test piece after 40% tensile deformation then release of the load. Thus, if the initial length of the test piece is L₀, and the length after 40% tensile deformation and release of the load is L₍ₜ₎, the recovery R₍ₜ₎ at time t from the release is equal to: 100×(1−((L₍ₜ₎−L₀)/L₀)/0.4).

Thus, if L₍ₜ₎=L₀, then R₍ₜ₎=100.

If L₍ₜ₎=1.4×L₀, then R₍ₜ₎=0.

The recovery test is carried out by first preparing a test piece approximately 200 μm thick, 6 cm long and 1 cm wide. If necessary, the test piece is optionally produced on a support film, the mechanical impact of which is judged to be small compared with the mechanical properties of the test piece.

The test piece is subjected to a tensile deformation of 40% of its length at a speed of 0.1 mm/s. The load is then released and 1 minute is allowed to pass.

The composition is preferably concentrated with respect to solids and consequently has a strong adherence, or a strong “tack”. The adherence can be measured as indicated below.

The tell “Fₘₐₓ” is intended to mean the maximum tensile force, measured using an extensometer, required to detach the respective surfaces, of 38 mm², of two rigid, inert, non-absorbent supports (A) and (B) placed opposite one another. The surface A is pre-coated with said composition in a proportion of 519 μg/mm², dried for 24 hours at 22° C. under a relative humidity of 50%. The surface B is uncoated. The two surfaces are then subjected, for 20 seconds, to a compression of 3 Newtons and finally subjected, for 30 seconds, to a tensile strain at a speed of 20 mm/minute. The tensile force Fₘₐₓ required to separate two surfaces of two rigid, inert and non-absorbent supports placed opposite one another, said surfaces being coated with the styling material to be evaluated, is determined using an extensometer, for example an apparatus of the Lloyd type, model LR5K. The rigid, inert and non-absorbent solid supports are typically a plastic of the glass type. According to the invention, the force Fₘₐₓ is preferentially greater than 1 N and preferably greater than 4 N.

Thus, the cosmetic composition comprises at least 20% by weight of solid(s), preferably at least 50% by weight of solid(s), relative to the total weight of the composition.

In particular, the cosmetic composition comprises at least one compound chosen from a wax, for example carnauba wax, beeswax and paraffin wax, an oil, a gum such as gum arabic, a modelling clay, a long-chain organic compound, such as cetyl phosphate, a polymer which has a T_{g} ranging from −20° C. to 20° C., and mixtures thereof.

In the present invention, the T_{g} (or glass transition temperature) values shown are theoretical T_{g} values determined from the theoretical T_{g} values of the constitutive monomers of the polymer, which can be found in a reference handbook, such as the Polymer Handbook, 3^{rd} ed, 1989, John Wiley, according to the following relationship, referred to as the Fox law:

$$\frac{1}{{T}_{g}} = {\sum }_{i} \frac{{ω}_{i}}{{T}_{{g}_{i}}}$$

ωi being the mass fraction of the monomer i in the considered sequence and T_{gi} being the glass transition temperature of the homopolymer of the monomer i.

Thus, unless otherwise specified, the T_{g} of polymers in the present application are theoretical T_{g} values.

Wax

Particularly suitable as waxes are those that were previously described.

In this variant, carnauba wax, beeswax and paraffin wax may be mentioned as particularly advantageous wax.

Oil

By way of oils suitable for the invention, mention may be made of:
hydrocarbon-based oils of animal origin,
hydrocarbon-based oils of plant origin, synthetic ethers containing from 10 to 40 carbon atoms, such as dicapryl ether,
synthetic esters, such as the oils of formula R₁COOR₂, in which R₁ represents a linear or branched fatty acid residue comprising from 1 to 40 carbon atoms and R₂ represents a hydrocarbon-based chain, which is especially branched, containing from 1 to 40 carbon atoms, on condition that R₁+R₂≧10. The esters may be chosen especially from fatty acid alcohol esters, for instance cetostearyl octanoate, isopropyl alcohol esters such as isopropyl myristate or isopropyl palmitate, ethyl palmitate, 2-ethylhexyl palmitate, isopropyl stearate, octyl stearate, hydroxylated esters, such as isostearyl lactate or octyl hydroxystearate, alkyl or polyalkyl ricinoleates, hexyl laurate, neopentanoic acid esters, such as isodecyl neopentanoate or isotridecyl neopentanoate, and isononanoic acid esters, such as isononyl isononanoate or isotridecyl isononanoate,
polyol esters and pentaerythritol esters, such as dipentaerythrityl tetrahydroxystearate/tetraisostearate,
fatty alcohols that are liquid at ambient temperature, with a branched and/or unsaturated carbon-based chain containing from 12 to 26 carbon atoms, such as 2-octyldodecanol, isostearyl alcohol and oleyl alcohol,
C₁₂-C₂₂ higher fatty acids, such as oleic acid, linoleic acid, linolenic acid, and mixtures thereof,
non-phenyl silicone oils, for instance caprylyl methicone, and
phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, and trimethyl-pentaphenyl-trisiloxane, and mixtures thereof; and also mixtures of these various oils.

Gum

By way of gum suitable for the present invention, mention may be made of gum arabic, gum tragacanth, cassia gum, camboge, shellac, karaya gum, tara gum or gellan gum.

In the context of the present invention, mention may be made of gum arabic as particularly advantageous gum.

Modelling Clay

By way of modelling clay suitable for the present invention, mention may be made of platicine, plastiline or else castilene. They are characterized by their malleability and the fact that they do not dry out and thus retain their malleability.

Long-Chain Organic Compound

By way of organic compound suitable for the present invention, mention may in particular be made of oils comprising long carbon-based chains, in particular of Cᵢₛ and more.

In the context of the present invention, mention may be made of cetyl phosphate as particularly advantageous long-chain organic compound.

Polymer Having a T_{g} Ranging from −20° C. to −20° C.

By way of polymer having a T_{g} ranging from −20° C. to 20° C. that is suitable for the present invention, mention may be made of polypropylenes, or else acrylic and methacrylic polymers, polyurethanes, polyester of higher Tg, but of which the Tg of the final material has been reduced through the use of a plasticizer.

In particular, mention may be made of the sulfonic polyester in particular sold under the name sulfonic polyester AQ 1350 by the company Eastman Chemicals, the Tg of which is in the region of 0° C.

The composition may, under pressure, deform and have the shape of the keratin fibre(s), in particular human keratin fibre(s), introduced into the mould cavity or cavities.

It deforms plastically under compression, i.e. it retains at least partly the deformation that it is given by the mould.

The device is preferably suitable for compression. Thus, the device may comprise one or more jaws that are deformable under compression. The jaw(s) may thus comprise at least one elastically deformable material, in particular an elastomeric material and/or a plastic.

The device may also comprise a mould which, by means of springs, can be compressed.

The mould may comprise a flexible material, in particular a plastic, especially an elastomeric material.

Colorant

In the assembly of variants A to D previously described in detail, it is advantageous for the composition to preferably comprise at least one colorant, which is in particular black in colour, or of another colour or several other colours.

New two-coloured or multicoloured effects may in particular be created.

The composition may be substantially the same colour as those of the additional fibres where appropriate.

This (or these) colorant(s) is (are) preferably chosen from pulverulent materials, liposoluble dyes and water-soluble dyes, and mixtures thereof.

Preferably, the compositions according to the invention comprise at least one pulverulent colorant. The pulverulent colorants may be chosen from pigments and nacres, and preferably from pigments.

The pigments may be white or coloured, inorganic and/or organic, and coated or uncoated. Among the inorganic pigments, mention may be made of metal oxides, in particular titanium dioxide, optionally surface-treated, zirconium, zinc or cerium oxide, and also iron, titanium or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with in particular ferric blue or chromium oxide, titanium mica with an organic pigment of the abovementioned type, and also nacreous pigments based on bismuth oxychloride. Examples of nacres that may also be mentioned include natural mica coated with titanium oxide, with iron oxide, with natural pigment or with bismuth oxychloride.

Among the commercially available nacres that may be mentioned are the nacres Timica, Flamenco and Duochrome (on mica base) sold by the company Engelhard, the Timiron nacres sold by the company Merck, the Prestige nacres on mica base sold by the company Eckart and the Sunshine nacres on synthetic mica base sold by the company Sun Chemical.

The nacres may more particularly have a yellow, pink, red, bronze, orange, brown, gold and/or coppery colour or tint.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

Preferably, the pigments contained in the compositions according to the invention are chosen from metal oxides.

A composition according to the invention may also comprise at least one particulate or non-particulate, water-soluble or water-insoluble colorant, preferably in a proportion of at least 0.01% by weight relative to the total weight of the composition.

For obvious reasons, this amount is liable to vary significantly with regard to the intensity of the desired colour effect and of the colour intensity afforded by the colorants under consideration, and its adjustment clearly falls within the competences of those skilled in the art.

These colorants may be present in a content ranging from 0.01% to 30% by weight relative to the total weight of the composition and in particular from 6% to 22% by weight relative to the total weight of the composition.

Preferably, the colorant(s) is (are) chosen from one or more metal oxides that are present in a content of greater than or equal to 2% by weight relative to the total weight of the composition, and advantageously inclusively between 6% and 22% by weight relative to the total weight of the composition.

XI. Additional Fibres

The assembly and the process according to the invention also make it possible to attach additional fibres to existing keratin fibres, in particular human keratin fibres, via the composition while limiting the visibility of the connection, thereby making it possible to give the keratin fibres, in particular human keratin fibres, a longer and/or thicker appearance.

Thus, the composition may be moulded onto at least one part of at least one of said keratin fibres, in particular human keratin fibres, and of at least one additional fibre, the composition ensuring the attachment of the additional fibre(s) to the keratin fibre(s), in particular human keratin fibre(s), in particular with the additional fibre(s) which extend the keratin fibre(s), in particular human keratin fibre(s), with or without axial overlap between a keratin fibre, in particular a human keratin fibre, and an additional fibre.

The composition may be deposited on just one part of the length of at least one of the keratin fibres, in particular human keratin fibres, and on just one part of the length of at least one additional fibre, better still on just one part of the length of each of the keratin fibres, in particular human keratin fibres, and on just one part of the length of each of the additional fibres.

The additional fibres may be covered with the composition only at their ends directed towards the keratin fibres, in particular human keratin fibres, preferably over a length of less than 10 millimetres, and preferably less than 5 mm.

The device may comprise one or more of said additional fibres, prepositioned in the cavity or cavities of the mould or interlinked with a support which makes it possible to preposition them. This support may be removable or resorbable, for example by washing with water.

The additional fibres may be synthetic fibres of the same colour as the keratin fibres of the user, and in particular may be darker in colour than the eyelashes of the user and in particular black. Typically, the fibres are 50 to 200 μm in diameter, and between 5 and 25 min in length, and can be tapered in order to resemble natural eyelashes.

At least one part of the composition, in particular the entire composition, may be deposited on the additional fibre(s) before they are introduced into the mould.

At least one part of the composition, in particular the entire composition, may be deposited on the additional fibre(s) while said fibres are present in the mould.

At least one part of the composition, in particular the entire composition, may be initially present in the mould before introduction of the additional fibres into the mould.

At least one part of the composition, in particular the entire composition, may be injected into the mould, via at least one injection channel.

The composition may be a composition according to one of the four variants of implementation of the invention previously described. The composition is preferably a cosmetic composition having a melting point of between 40° C. and 120° C., and comprising at least 15% by weight of meltable compound(s), relative to the total weight of the composition.

Heat then makes it possible to attach the additional fibres to the keratin fibres, in particular human keratin fibres.

According to another preferred embodiment variant, the cosmetic composition is fluid at ambient temperature and contains at least particles in the dispersed state in a liquid medium.

The device may comprise two jaws, at least one of the jaws comprising a heating element.

The device may comprise a light source and two jaws, at least one of the two jaws being transparent to at least a part of the radiation emitted by the light source, such that said radiation reaches the composition.

The keratin fibres, in particular human keratin fibres, may be cut prior to their introduction into the mould, in particular in order to give them a length which will make them fit to the arrangement of the additional fibres in the mould.

The invention may be better understood from reading the following detailed description of non-limiting illustrative embodiments thereof and from examining the appended drawings, in which:

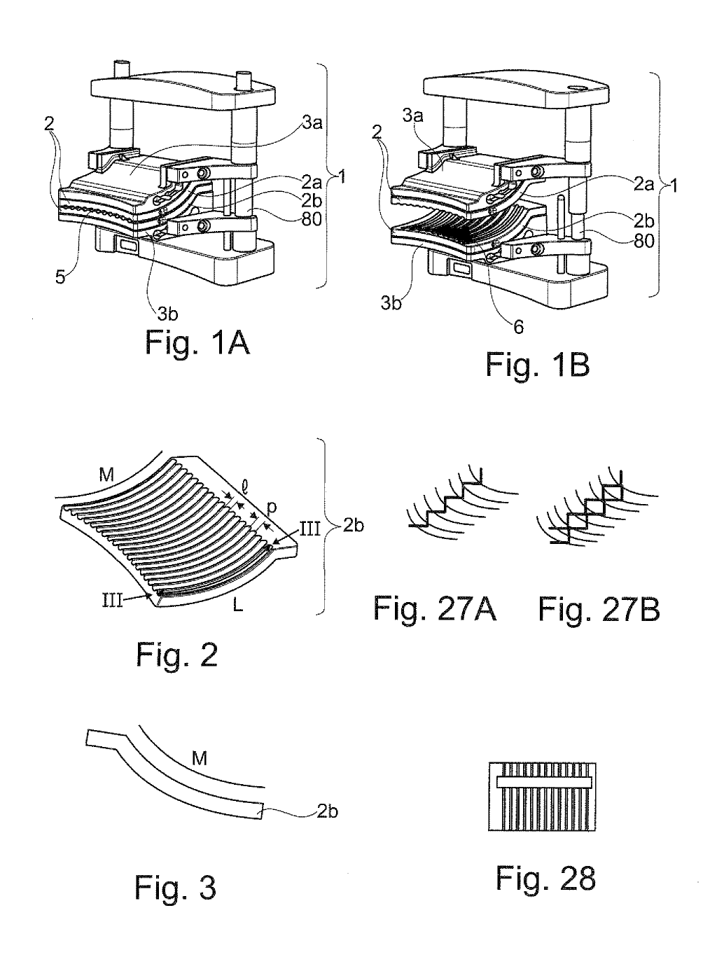

FIGS. 1A and 1B represent an example of an assembly according to the invention, respectively with the jaws of the mould closed in moulding configuration and open,

FIG. 2 represents in isolation one of the parts of the mould of FIGS. 1A and 1B,

FIG. 3 represents, in section along III of FIG. 2, a part of the mould of this figure,

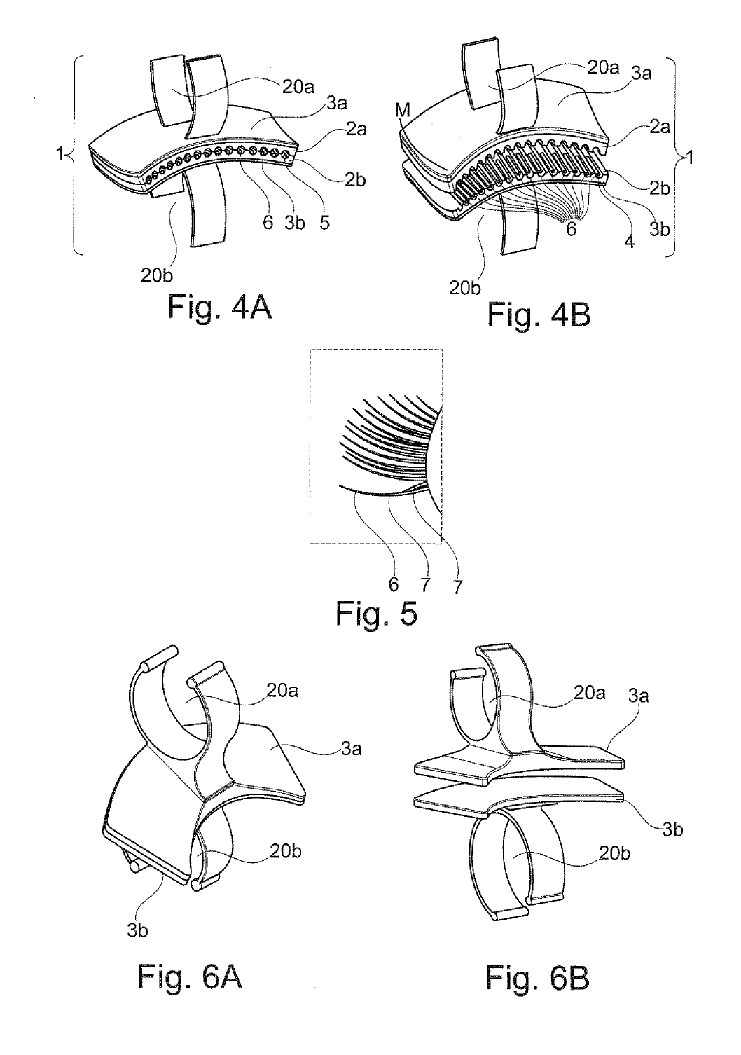

FIGS. 4A and 4B represent another example of an assembly according to the invention, in the form of a clamp, respectively with the jaws of the mould closed in moulding configuration and open,

FIG. 5 illustrates the deposit of composition obtained on eyelashes after cosmetic treatment,

FIGS. 6A and 6B, 8A and 8B, 11 and 10 represent other examples of devices according to the invention in the form of a clamp, the moulds not being represented, FIGS. 6A and 6B illustrating the same example of a device respectively with the jaws of the mould closed and open, FIGS. 8A and 8B illustrating the same example of a device respectively with the jaws of the mould closed and open,

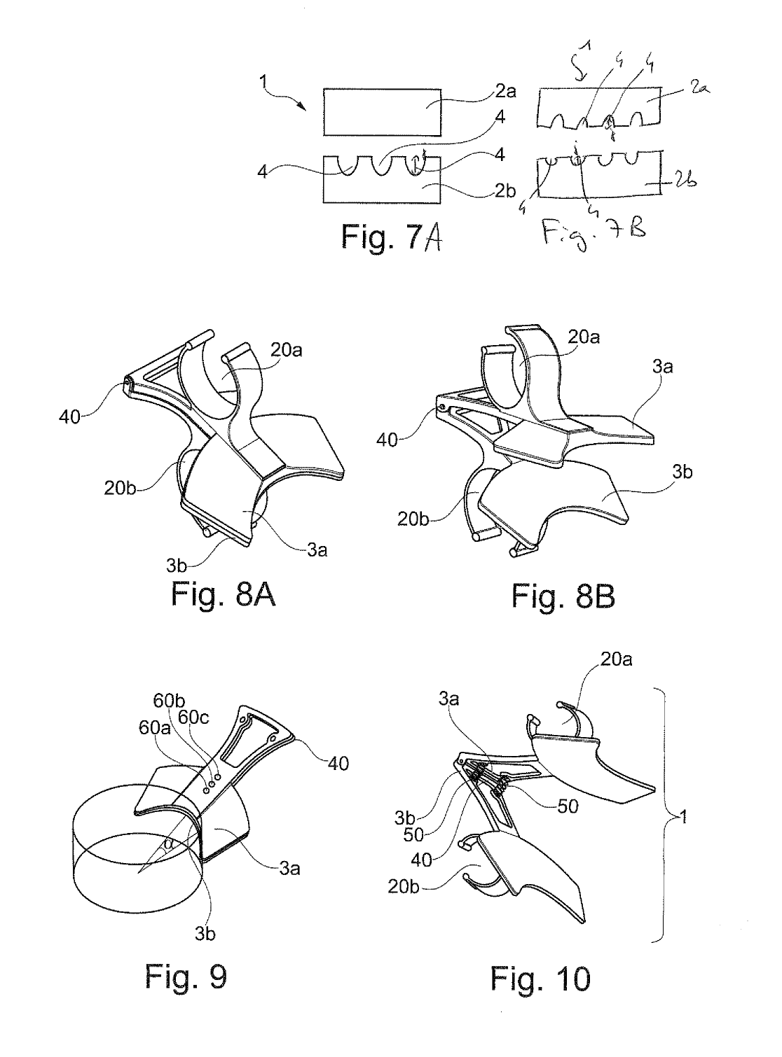

FIGS. 7A and 7B represent two examples of a mould before closing,

FIG. 9 represents the device from FIG. 8A, the housings having been dismantled,

FIGS. 12A and 12B represent various steps for using an example of a demoulding system,

FIG. 13 represents another example of a mould before closing,

FIG. 14 represents an example of a part of a device equipped with blades,

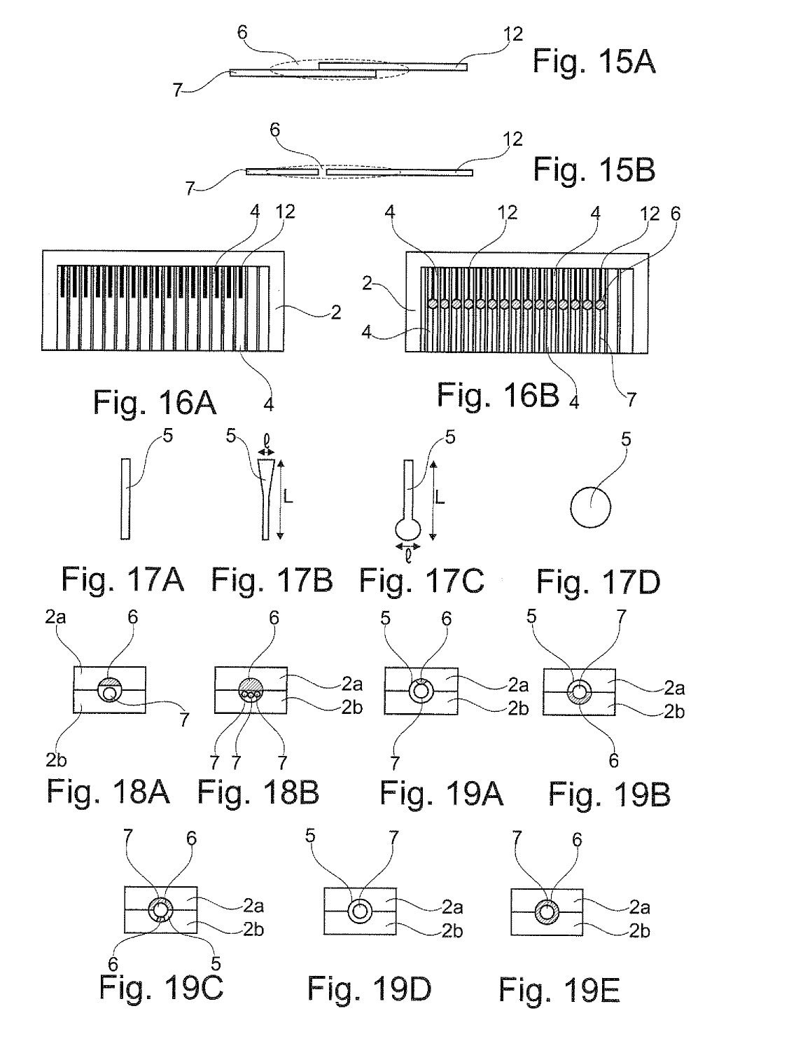

FIGS. 15A and 15B represent two examples of connection of an additional fibre to an eyelash using a composition according to the invention,

FIGS. 16A and 16B illustrate an example of attachment of additional fibres to eyelashes,

FIGS. 17A to 17D represent, in section, examples of mould cavity shape,

FIGS. 18A and 18B represent respectively a cavity comprising an eyelash and a cavity comprising several eyelashes,

FIGS. 19A to 19E represent, in section, examples of arrangements of the composition according to the invention before moulding, within a mould cavity into which an eyelash is introduced,

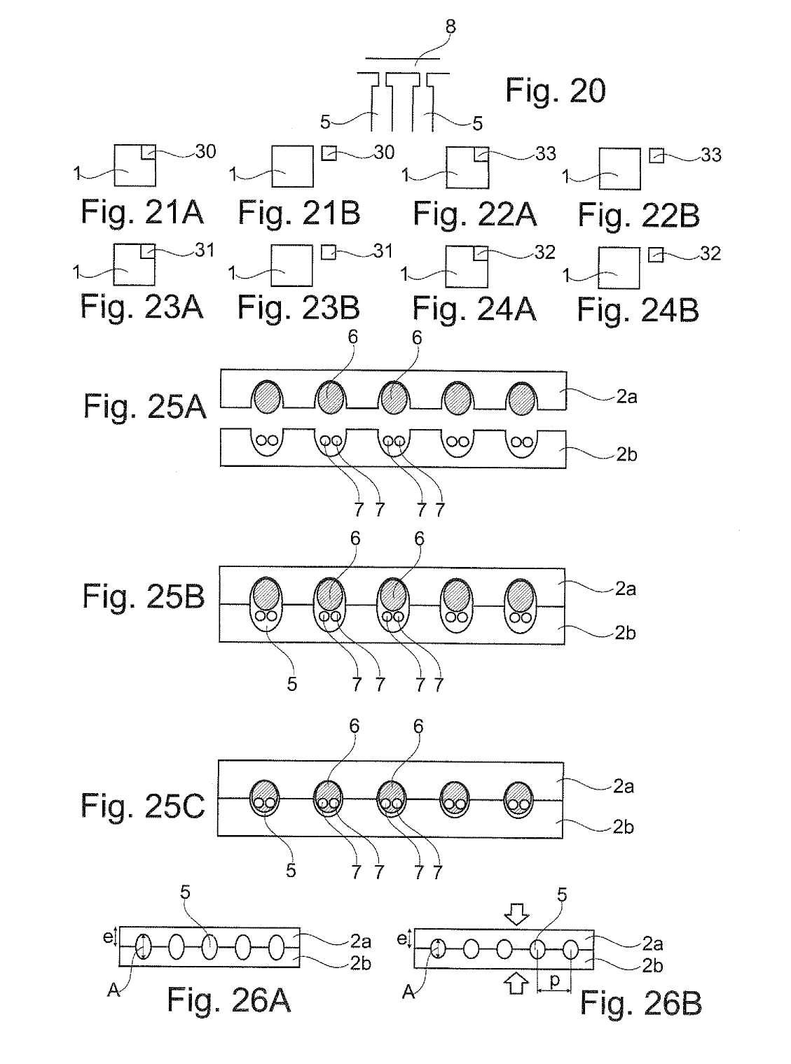

FIG. 20 represents an example of an injection channel communicating with mould cavities,

FIGS. 21A and 21B represent, very diagrammatically, respectively a device according to the invention comprising a heating element and a device according to the invention placed in proximity to a heating element,

FIGS. 22A and 22B represent, very diagrammatically, respectively a device according to the invention comprising a cooling system and a device according to the invention placed in proximity to a cooling system,

FIGS. 23A and 23B represent, very diagrammatically, respectively a device according to the invention comprising a system which assists drying and a device according to the invention placed in proximity to a system which assists drying,

FIGS. 24A and 24B represent, very diagrammatically, respectively a device according to the invention comprising a light element and a device according to the invention placed in proximity to a light element,

FIGS. 25A, 25B and 25C illustrate various steps of an example of moulding by compression,

FIGS. 26A and 26B illustrate an example of mould compression,

FIGS. 27A and 27B are examples of patterns formed with the composition on the eyelashes, and

FIG. 28 is an example of an imprint with a transverse groove.

FIGS. 1A and 1B represent an example of an assembly according to the invention comprising a device 1 and a cosmetic composition 6 for cosmetic treatment of eyelashes.

The device 1 comprises a mould 2 which has two parts 2a and 2b. The mould 2 preferably comprises an elastomeric material. The two parts 2a and 2b of the mould 2 each have a thickness e of approximately 2 mm for example.

The two parts 2a and 2b of the mould 2 comprise imprints 4, in the form of grooves of elongated shape. Each part 2a, 2b comprises, for example, as illustrated, 17 imprints 4, for example of width l of approximately 0.5 mm, of length L of approximately 2.5 cm and of approximately semicircular cross section. The imprints 4 of each part 2a, 2b of the mould 2 are preferably substantially parallel to one another. The imprints are also preferably, as illustrated, evenly spaced out, according to a step p of 1.5 mm, centre to centre.

The device 1 comprises two jaws 3a and 3b which carry respectively the mould parts 2a and 2b.

The two parts of the mould 2 and the jaws 3a and 3b may have additional reliefs, so as to be able to accurately place the two parts 2a, 2b of the mould 2 on each jaw 3a, 3b.

The lower jaw 3b comprises, in the example under consideration, a temperature-controlled heating element 30.

The lower jaw 3b is fixed, whereas the upper jaw 3a slides vertically by means of a guidance system 80.

When the jaws 3a and 3b are closed against one another, as represented in FIG. 1A, cavities 5 are formed by virtue of the imprints 4, which are superimposed in pairs.

In the example under consideration, 17 cavities of elongated shape, having a width l of approximately 0.5 mm, a length L of approximately 2.5 cm and an approximately circular cross section, which are substantially parallel to one another and evenly spaced out, according to a step p of 1.5 mm, centre to centre, are thus created.

The cavities 5 are closed on the sides and open out to the exterior, on the side where the eyelashes are introduced, via a front opening through which the eyelashes are introduced. The cavities are closed at their rear end.

To use the device 1, the cosmetic composition 6 is, for example, placed in imprints 4 of the part 2b of the mould 2, mounted on the lower jaw, before introduction of the eyelashes. The composition 6 is preferably a cosmetic composition having a melting point of between 40° C. and 120° C., and comprising at least 15% by weight of meltable compound(s), relative to the total weight of the composition, as previously described.

The eyelashes are introduced into the imprints 4 of the mould 2 of the device 1 in its open form (FIG. 1B), then the jaws 3a, 3b are closed so as to hold the eyelashes in the cavities 5.

The heating element 30 brings the composition 6 to a temperature greater than its melting point, in such a way that the composition 6 takes, in the fluid state, the shape of the imprints 4. The moulding of the composition onto the eyelashes is carried out in situ in the cavities 5 of the mould 2.

Then, the heating element 30 stops heating the composition and said composition is left to cool.

The jaws 3a, 3b are opened and the eyelashes are withdrawn when the temperature of the composition 6 has come back down below its melting point. The device 1 can be produced such that the closing of the jaws 3a and 3b automatically triggers the heating of the composition 6, and so that the heating is automatically stopped when the desired temperature is reached.

The opening of the jaws 3a and 3b can take place automatically, as appropriate. As a variant, a sound or light signal can indicate that the jaws can be opened.

FIGS. 2 and 3 represent more particularly the part 2b of the mould 2 of FIGS. 1A and 1B.

It is seen that the part 2b can be curved along the longitudinal direction M of the imprints 4, in order to reproduce the curved shape of the eyelashes, the radius of curvature preferably being between 15 and 25 mm.

The edge of the part 2b intended to come into contact with the eyelid may have a rounded shape, concave towards the exterior, with a radius of curvature of preferably between 15 and 25 mm.

The device 1 may, according to one implementation variant of the invention, be in the form of a clamp, as represented in FIGS. 4A and 4B.

Like the device represented in FIGS. 1A and 1B, this device in the form of a clamp comprises a mould 2 in two parts 2a and 2b. The mould 2 preferably comprises an elastomeric material. The two parts 2a and 2b of the mould 2 each have, for example, a thickness e of approximately 2 mm. The two parts 2a and 2b of the mould 2 comprise imprints 4, in the form of grooves of elongated shape. The device 1 comprises two jaws 3a and 3b which carry respectively the mould parts 2a and 2b.

Each part 2a, 2b comprises, for example, 15 imprints 4, for example having a width l of approximately 0.5 mm, a length L of approximately 2.5 cm, and a decreasing semicircular cross section so as to give the eyelashes, once moulded with the composition 6, a tapered appearance. The imprints 4 of each part 2a, 2b of the mould 2 are preferably, as illustrated, substantially parallel to one another. The imprints are evenly spaced out, according to a step p of 1.5 mm, centre to centre.

The mould 2a, 2b may have a radius of curvature along the direction M of FIG. 4B of between 15 and 25 mm in order to follow the shape of the eyelashes. The edge of the part 2b intended to come into contact with the eyelid may have a rounded shape, concave towards the exterior, with a radius of curvature of preferably between 15 and 25 mm.

The edge of the jaws 3a, 3b and of the mould 2a, 2b intended to come into contact with the eyelid may have a rounded shape, concave towards the exterior, in particular circular, the edge of the eyelid describing, to a first approximation, an arc of a circle.

When the jaws are closed against one another, as represented in FIG. 4A, cavities 5 are formed by virtue of the imprints 4, which are superimposed in pairs. 15 cavities 5 of elongated shape, having a width l of approximately 0.5 mm, a length L of approximately 2.5 cm and a circular cross section of decreasing diameter as the distance from the opening for introducing the eyelashes increases, which are substantially parallel to one another and evenly spaced out, according to a step p of 1.5 mm, centre to centre, are thus, for example, created. The cavities 5 are closed on the sides and open out to the exterior, on the front side where the eyelashes are introduced, via a circular opening through which the eyelashes are introduced. The cavities are closed at their rear end.

The device 1 in the form of a clamp may comprise, as illustrated, two housings 20a, 20b into which fingers of one hand, for example the thumb and the index finger, can be introduced so as to move the two jaws 3a, 3b apart, in order to make it possible to introduce the eyelashes between them and to close them on said eyelashes.

The cosmetic composition 6 to be moulded is placed in imprints 4 of the part 2b of the mould 2 before introduction of the eyelashes. The composition 6 is preferably a cosmetic composition having a melting point of between 40° C. and 120° C., and comprising at least 15% by weight of meltable compound(s), relative to the total weight of the composition, as previously described.

An independent oven, not represented, can be used to bring the composition to a temperature above its melting point, before introduction of the eyelashes into the imprints 4 of the mould. The housings 20a, 20b preferably remain cold, the heating preferably taking place by radiative transfer on the faces of the mould having the imprints.

The cavity of the oven may have a parallelepipedal shape, for example an approximately 10 cm-sided parallelepipedal shape. In addition to its heating function, the oven can also make it possible to present the jaws in such a way that the user can seize them directly between the thumb and index finger, in a single action. In particular, it is possible to use portable clamps of which the internal faces are placed in front of the heat sources and the external faces bear grasping rings adjusted to the shape of the fingers.

The surfaces carrying the composition can, for example, be heated in the range 30° C.−80° C. The oven can be powered by a low-voltage power source.

The heating temperature can be adjustable by the user. A casing can enable a temperature setting to be displayed. The heating power can be about 5 W for example.

When the composition is at a temperature above its melting point, the user's thumb and index finger are introduced into the housings 20a and 20b, then the device 1 is brought into proximity to the eyelashes and the latter are introduced into the imprints 4 of the mould 2 of the device 1 in its open form (FIG. 4B), then the jaws 3a, 3b are closed so as to hold the eyelashes in the cavities 5.

The moulding is carried out in situ in the cavity or cavities 5 of the mould 2.

The jaws 3a, 3b are then opened and the eyelashes are withdrawn when the temperature of the composition 6 has come back down below its melting point.

An example of the result of the cosmetic treatment of the eyelashes 7 by such a device 1 is illustrated in FIG. 5.

The device 1 in the form of a clamp may be devoid of a system for guiding the movement of one jaw relative to the other, as represented in FIGS. 6A and 6B. This device has two housings 20a, 20b for the fingers, like the example previously described.

Only one of the parts can have imprints, which is particularly advantageous for a device 1 devoid of a guidance system, since the bringing together of the two parts of the mould does not need to be carried out very accurately.

By way of example, represented in FIG. 7A is a device 1 in which the part 2a of the mould 2 may be smooth and the part 2b may comprise semicircular imprints 4.

Moreover, each cavity 5 may have an elongated shape which does not reproduce the curvature of the eyelash(es) introduced, it being possible in particular for the cavity to be non-curved along its longitudinal direction, having, for example, a rectilinear longitudinal axis.

The device 1 in the form of a clamp may comprise a guidance system, for example in the form of reliefs having complementary shapes, which brings the jaws 3a, 3b into position so that the two parts of the mould 2a, 2b accurately correspond to one another. The device 1 in the form of a clamp may thus comprise, for example, a male and female cone guidance system.

The device 1 in the form of a clamp may comprise a guidance system of film hinge type or other form of articulation 40, as represented in FIGS. 8A and 8B, such that the jaws can be moved together by a pivoting movement with respect to one another. Each cavity formed by the superimposition of two imprints may have a rectilinear longitudinal axis. The front edges of the jaws 3a, 3b intended to come into contact with the eyelid may have a circular shape, as illustrated.

During the use of the device 1, the angle α made by the plane of moulding with the horizontal may be zero or non-zero, in particular between 20 and 40°, as illustrated in FIG. 9. Inclining the plane of the moulds makes it possible to push the curvature of the edge coming into contact with the eyelid as close as possible to the curvature of the eyelid itself.

The hinge 40 may comprise one or more springs 50 as represented in FIG. 10, which assist the opening movement for example.

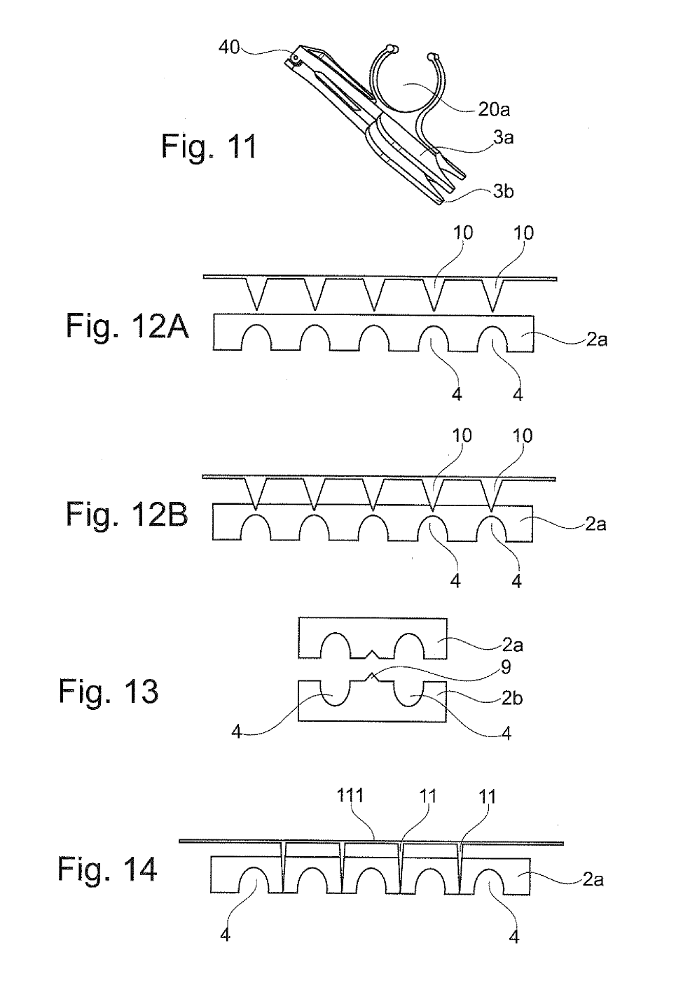

The device 1 in the form of a clamp may comprise a single housing 20a for receiving a finger, as illustrated in FIG. 11, or two housings, as previously described.

It may be possible for the housing(s) 20a, 20b to be oriented rotationally, which enables an adjustment according to the angle between the user's thumb and index finger. They may be positionable, where appropriate, at various points 60a, 60b or 60c, depending on the desired distance between the fingers and the face of the user, as illustrated in FIG. 9.

The mould 2a, 2b may be integrated into the jaws 3a, 3b. The device 1 in the form of a clamp may then be made of a flexible material of silicone type for example, in order to facilitate demoulding.

The mould may also be made of a flexible material of silicone type with the inclusion of a rigid frame. The housing(s) 20a, 20b may be made of a rigid material, in order to improve the holding of the clamps in place and the overall shape of the mould, while facilitating demoulding.

It is possible for the mould 2a, 2b not to be integrated into the jaws 3a, 3b, the jaws carrying the mould, which is then added to the jaws. The mould may be flexible and held on the jaws for example by adherence or by matching shapes, for example by click-fastening and/or sliding attachment. The jaws are then preferably rigid.

The mould 2a, 2b may or may not be disposable. The jaws 3a, 3b may or may not be reusable.

The composition 6 may be present within the mould before use of the device 1, in the part 2a, the part 2b or in both parts, in particular in the form of a pulverulent composition. The loading of the mould with composition may be carried out prior to the use of the device, for example using a metering device.

The device in the form of a clamp has the advantage of being light, of allowing the user to precisely perceive the stresses applied, to the eyelid, during the operations, of creating a very small visual eclipse allowing optimal vision for the user, and of being easy to use.

The device 1, whether or not it is in the form of a clamp, may comprise a system which facilitates demoulding, for instance a set of small blades 10 which deform the mould, for example when moved relative to said mould in the direction of the imprints 4, as illustrated in FIGS. 12A and 12B.

The mould 2 may also comprise, as illustrated in FIG. 13, reliefs 9 between two imprints 4 intended to form two cavities, so as to cut any bridges of composition.

The device 1 may comprise blades 11 which serve to cut any bridges of composition between two cavities, as illustrated in FIG. 14.

The blades 11 are, for example, retracted within the part of the mould 2a before closing thereof, and tend to advance upon closing of the mould. The blades 11 are, for example, interlinked with a support 111 which is mobile relative to the part 2a.

As a variant, the material of the part 2a is elastomeric and overmoulded on the blades 11, which can push on the bridges of composition extending between the imprints 4 by virtue of the deformability of the material with which the part 2a is made.

Other systems can be further used for cutting the bridges, for example a system which makes it possible to send compressed air via slits located between the imprints or a heating element.

The composition 6 can make it possible to attach an additional fibre 12 to an eyelash 7, with or without axial overlap between the eyelash and the additional fibre, as illustrated respectively in FIGS. 15A and 15B. The eyelash may be on the additional fibre if the additional fibre is in a cavity of the lower mould part, or vice versa, if the additional fibres are in the cavities of the upper mould part. In practice, the additional fibres may also be next to the eyelashes depending on the organization caused by the pressure of the two parts of the mould.

The additional fibres may in particular be false eyelashes.

The additional fibres 12 may be placed on hold in the imprints 4 of the mould 2 (FIG. 16A). The composition 6 may then be applied to the ends of the additional fibres 12 and the eyelashes 7 introduced into the imprints 4 of the mould 2 and brought into contact with the composition 6 (FIG. 16B).

The cavities 5 may, according to various variants, have a shape which is approximately cylindrical (FIGS. 17A and 7B), approximately spherical (FIG. 17D), cylindrical on a part of their length (L) and spherical on the other part of their length (FIG. 17C), or have a decreasing cross section so as to give the eyelash(es), once moulded by the composition, a tapered appearance (FIG. 17B).

They may also have a semi-cylindrical shape in the case where the part 2a of the mould is flat and the part 2b of the mould is semi-cylindrical in the cavity 5 formation zone (FIG. 7A).

The depth (j) of the imprints 4 may range between 75 μm and 1.5 mm.

Each cavity 5 can receive one or more eyelashes 7, as illustrated respectively in FIGS. 18A and 18B.

Before moulding, the composition 6 may, according to various variants, be present on only one part of the cavity 5, as represented in FIGS. 19A to 19C, on all of the cavity, as illustrated in FIG. 19E, or absent from the cavity as represented in FIG. 19D. In the latter case, the composition may be injected via an injection channel 8 communicating with the cavities 5 of the mould, as illustrated in FIG. 20.

The composition 6 may be present initially, before putting the eyelashes in place, on the upper part of the cavity, corresponding to the part 2a of the mould (FIG. 19A), on the lower part of the cavity, corresponding to the part 2b of the mould (FIG. 19B), or both on the upper part and on the lower part of the cavity, corresponding to both parts 2a and 2b of the mould (FIGS. 19C and 19E).

As previously described with reference to FIGS. 1A and 1B, 4A and 4B, a composition 6 which is a cosmetic composition having a melting point of between 40° C. and 120° C., and comprising at least 15% by weight of meltable compound(s), relative to the total weight of the composition, is preferably used.

The device 1 may then comprise, or be placed in proximity to, a heating element 30, as illustrated very diagrammatically respectively in FIGS. 21A and 21B, in order to melt the composition 6, the heating element 30 being, for example, a resistive element, a radiative element or a wave dispenser. The device may provide for the injection of the composition in molten form, in particular via an injection channel 8, as represented in FIG. 20.

The device may comprise, or be placed in proximity to, a cooling system 33, which cools for example by Peltier effect, as represented respectively in FIGS. 22A and 22B, in order to accelerate the cooling of the composition and the demoulding.

According to another example of implementation of the invention, it is possible to replace the composition 6 having a melting point of between 40° C. and 120° C. with a cosmetic composition 6 which is fluid at ambient temperature and contains at least particles in the dispersed state in a liquid medium.

The device may then comprise, or be placed in proximity to, a heating element 30, as represented respectively in FIGS. 21A and 21B, the heating element being, for example, a resistive element, a radiative element or a wave dispenser.

The device may comprise, or be placed in proximity to, a system 31 which assists drying by providing energy, in particular in the form of light or heat, and/or which comprises an aeration circuit, suction and/or blowing of air, as illustrated respectively in FIGS. 23A and 23B, in order to evaporate at least a part of the liquid medium.

The mould 2 may comprise an absorber specific for the liquid medium in order to eliminate at least a part of the liquid medium.

In another example of implementation of the invention, a composition 6 which is a composition which is fluid at ambient temperature and contains at least one compound that is activatable by a physical or chemical stimulus is used. The eyelashes and the composition are enclosed in the mould. After reaction, demoulding is performed.

The device may then comprise, or be placed in proximity to, a heating element 30, as represented respectively in FIGS. 21A and 21B, in order to accelerate the reaction, the heating element 30 being, for example, a resistive element, a radiative element or a wave dispenser.

The device 1 according to the invention may comprise a triggering system, such as a light element 32, as represented respectively in FIGS. 24A and 24B, in order to trigger the reaction, for example an IR, UV or visible light illuminator. The mould 2 is then preferably transparent to the wavelengths used to trigger the reaction.

In another example of implementation of the invention, the composition 6 is a ductile composition and preferably has a Young's modulus of less than or equal to 100 MPa at ambient temperature and pressure.

FIGS. 25A to 25C illustrate various steps of an example of moulding by compression. Composition 6 is initially present in the part 2a of the mould and the eyelashes 7 in the other part 2b.

After closure of the two parts of the mould, the cavities 5 are formed (FIGS. 25B and 25C).

A compression exerted on the mould, which is flexible, makes it possible to reduce the volume of the cavities of the mould (FIG. 25C) and to enhance the contact between the eyelashes 7 and the composition 6.

FIGS. 26A and 26B represent a mould before and after compression of the cavities, without the eyelashes or the composition. The largest dimension of the cavities A is, for example, 3 mm before compression (FIG. 26A) and it is reduced by at least 30%, for example to 2 mm, after compression (FIG. 26B).

FIGS. 27A and 27B represent examples of patterns formed by the composition on the eyelashes; in the example of FIG. 27A, the composition forms waves transversely to the eyelashes, and in the example of FIG. 27B, the composition forms a grid.

FIG. 28 represents a mould imprint with a groove transverse to those intended to receive the eyelashes or the additional fibres. The transverse groove is, for example, 4 mm wide and 1 mm deep. The transverse groove can be produced 5 mm from the edge. The imprint of FIG. 28 can be used by placing the composition in the transverse groove and the additional fibres in the parallel grooves.

### EXAMPLES

### Examples 1 to 8: Composition Having a Melting Point of Between 40° C. and 120° C.

### Example 1: Assembly Containing a Composition Based on a Thermoplastic Polymer and a Device which has a Heating Element

1) Cosmetic Composition

A composition in accordance with the invention is prepared as described below.

All the starting materials used are weighed out using a balance (accuracy 0.01 g). The compounds are melted in a jacketed 500 ml heating pan with a circulation of hot oil to control the temperature. The assembly is heated to approximately 95-98° C.

Once the mixture has melted, it is homogenized by stirring using a Moritz blender (stirring of rotor stator type consisting of a fixed part in which a mobile second part rotates at variable speed), in order to disperse the pigments.

The composition is then used hot in liquid form to be deposited on the moulds or at ambient temperature in solid form.

The formulation is prepared using the weight proportions described below.

| | % by weight |
|---|---|
| | (relative to the |
| | total weight of the |
| Compounds | composition) |
| Mixture of copolymer of ethylene-vinyl acetate and of | 90 |
| paraffin sold under the name COOL BIND | |
| 34-1300 ® by the company National Starch | |
| Iron oxide sold under the name SUNPURO BLACK | 10 |
| IRON OXIDE C33-7001 by the company SUN | |

2) Device

The device used in Example 1 is as described in FIGS. 1A and 1B.

It contains two mobile jaws (3a) and (3b), one of the two jaws having a temperature-controlled heating system.

The two parts (2a) and (2b) of the mould, placed between the two jaws, are made of crosslinked silicone elastomer and are 2 mm thick. They each comprise 20 grooves approximately 0.5 mm in diameter over a length of 2.5 cm.

The two jaws comprise two lugs and the two parts of the mould each comprise two housings which fit the lugs.

When the two parts of the mould are brought together, 20 leaktight cavities, closed at their end, are created on the sides.

3) Cosmetic Assembly

The thermoplastic polymer-based molten cosmetic composition is placed in the cavities and grooves of the two parts (2a) and (2b) of the mould using a pipette. The assembly is then left to cool.

The two parts (2a) and (2b) of the mould are then placed on the jaws (3a) and (3b) of the device (1).

The two jaws (3a) and (3b) are moved near to the eyelash fringe and then closed so as to trap it.

The heating is activated by virtue of the heating element of the device (1) for 4 minutes until a temperature of approximately 80° C. is reached within the composition.

The heating is then stopped and the assembly is left to cool for 4 minutes. At the end of the 4 minutes, the two jaws (3a) and (3b) are moved apart.

The two parts (2a) and (2b) of the mould remain attached to the eyelash fringe. The two parts of the mould are therefore moved apart, by pulling along their width, in order to release the eyelashes.

The result is then slightly rubbed in order to break the possible bridges of composition formed between two imprints.

A homogeneous makeup result is obtained on the eyelashes, without any eyelash brushing movement. The makeup is easy to apply without any need to calibrate the amount of composition taken and applied.

The term “homogeneous makeup result” is thus intended to mean, on the one hand, a greater volume by increasing the thickness of the eyelashes and/or increasing the diameter of the eyelashes, and/or an extension of the eyelashes by positioning of the sleeves of compositions moulded in the continuity of the free ends of the eyelashes constituting the eyelash fringe.

### Example 2: Assembly Containing a Wax-Based Composition and a Device which has a Heating Element

1) Cosmetic Composition

A composition in accordance with the invention is prepared as described below.

All the starting materials used are weighed out using a balance (accuracy 0.01 g). The compounds are melted in a jacketed 500 ml heating pan with a circulation of hot oil to control the temperature. The assembly is heated to approximately 95-98° C.

Once the mixture has melted, it is homogenized by stirring using a Moritz blender (stirring of rotor stator type consisting of a fixed part in which a mobile second part rotates at variable speed), in order to disperse the pigments.

The composition is then used hot in liquid form to be deposited on the moulds or at ambient temperature in solid form.

The formulation is prepared using the weight proportions described below.

| | % by weight (relative |
|---|---|
| | to the total weight of |
| Compounds | the composition) |
| Beeswax sold under the name White Beeswax | 90 |
| SP-453P by the company Strahl & Pitsch | |
| Iron oxide sold under the name SUNPURO | 10 |
| BLACK IRON OXIDE C33-7001 by the | |
| company SUN | |

2) Device

The device used is identical to that of Example 1.

3) Cosmetic Assembly

The wax-based cosmetic composition is placed in the cavities and grooves of the two parts (2a) and (2b) of the mould.

The device and the cosmetic composition are used in the same way as illustrated in Example 1.

A homogeneous makeup result is obtained on the eyelashes, without any eyelash brushing movement. The makeup is easy to apply without any need to calibrate the amount of composition taken and applied.

### Example 3: Assembly Containing a Composition Based on a Semi-Crystalline Polymer and a Device which has a Heating Element

1) Cosmetic Composition

A composition in accordance with the invention is prepared as described in Example 2.

The formulation is prepared using the weight proportions described below.

| | % by weight (relative |
|---|---|
| | to the total weight of |
| Compounds | the composition) |
| Poly(C₁₀-C₃₀)alkyl acrylate sold under the name | 85 |
| Intelimer IPA 13-1 NG by the company Air | |
| products and Chemical | |
| Iron oxide sold under the name SUNPURO | 15 |
| BLACK IRON OXIDE C33-7001 by the | |
| company SUN | |

2) Device

The device used is identical to that of Example 1.

3) Cosmetic Assembly

The cosmetic composition based on a semi-crystalline polymer is placed in the cavities and grooves of the two parts (2a) and (2b) of the mould.

The device and the cosmetic composition are used in the same way as illustrated in Example 1.

A homogeneous makeup result is obtained on the eyelashes, without any eyelash brushing movement. The makeup is easy to apply without any need to calibrate the amount of composition taken and applied.

### Example 4: Assembly Containing a Composition Based on a Thermoplastic Polymer and a Device with No Heating Element

1) Cosmetic Composition

A composition as described in Example 1 is prepared.

2) Device

The device used in Example 4 is as described in FIGS. 4A and 4B.

It consists of a clamp containing two mobile jaws (3a) and (3b).

The two parts (2a) and (2b) of the mould, placed between the two jaws, are made of crosslinked silicone elastomer and are 2 mm thick. They each comprise 20 grooves approximately 0.5 mm in diameter over a length of 2.5 cm.

The two jaws comprise two lugs and the two parts of the mould each comprise two housings which fit the lugs.

When the two parts of the mould are brought together, 20 leaktight cavities, closed at their end, are created on the sides.

3) Cosmetic Assembly

The cosmetic composition based on a thermoplastic polymer is placed in the cavities and grooves of the two parts (2a) and (2b) of the mould.

The composition is melted by means of an external heat source.

The assembly is placed in proximity to a heat source, which heats the two parts of the mould.

The oven is based on a heating system comprising high-luminosity 5600K white LEDs. The LEDs are divided up into 6 networks of 14 LEDs: model Bridgelux BXRA-56C1100-B-00. The total light flux delivered is 8700 lumen. The LEDs produce 124 Lm/W@5600° K. The oven comprises a forced ventilation system with air intakes via the bottom and evacuation holes on the flanks. The LEDs are on a copper heat pipe mounted on a finned radiator, itself cooled by a fan. A parabolic deflector homogenizes the light on the moulds to be heated. The clamp comprises two jaws.

The oven is controlled by a control unit which supplies the oven with a Traco Power 100 W-24 V power supply. It makes it possible to adjust the temperature setting and manages the temperature servo-control. The temperature management is carried out using the ESM-4420 PID (proportional integral derivative) temperature controller.

The assembly makes it possible to produce a heat of approximately 70° C. and rapid heat transfer.

Removed from the oven, the eyelashes are clasped in said assembly which is then cooled for 5 minutes, then demoulded.

The result is then slightly rubbed in order to break the possible bridges of composition formed between two imprints.

A homogeneous makeup result is obtained on the eyelashes, without any eyelash brushing movement. The makeup is easy to apply without any need to calibrate the amount of composition taken and applied.

### Example 5: Assembly Containing a Fluid Composition and a Device which has a Heating Element

1) Fluid Cosmetic Composition

The fluid cosmetic composition used is identical to that sold under the name Mascara Volume Millions de Cils by the company L'Oreal. This composition has a viscosity of approximately 4 Pa·s.

2) Device

The device used is identical to that of Example 1.

3) Cosmetic Assembly

The cosmetic composition is placed in the cavities and grooves of the two parts (2a) and (2b) of the mould.

The device and the cosmetic composition are used in the same way as illustrated in Example 1.

A homogeneous makeup result is obtained on the eyelashes, without any eyelash brushing movement. The makeup is easy to apply without any need to calibrate the amount of composition taken and applied.

### Example 6: Assembly Containing Additional Fibres

The cosmetic composition and the device are the same as those used in Example 1.

Additional fibres 1 cm in length are placed in the imprints of one of the two parts of the mould, as illustrated in FIG. 16A.

The composition is melted and then one drop of the composition is placed, with a hot micropipette, at each end of the additional fibres, as represented in FIG. 16B.

After cooling of the composition, one part of the mould thus prepared is mounted on one jaw and one part of the unused mould is mounted on the other jaw.

The assembly comprising the fibres is heated for 5 minutes in an oven produced as follows.

The oven is based on a heating system comprising high-luminosity 5600K white LEDs. The LEDs are divided up into 6 networks of 14 LEDs: model Bridgelux BXRA-56C1100-B-00. The total light flux delivered is 8700 lumen. The LEDs produce 124 Lm/W@5600° K. The oven comprises a forced ventilation system with air intakes via the bottom and evacuation holes on the flanks. The LEDs are on a copper heat pipe mounted on a finned radiator, itself cooled by a fan. A parabolic deflector homogenizes the light on the moulds to be heated. The clamp comprises two jaws.

The oven is controlled by a control unit which supplies the oven with a Traco Power 100 W-24 V power supply. It makes it possible to adjust the temperature setting and manages the temperature servo-control. The temperature management is carried out using the ESM-4420 PID (proportional integral derivative) temperature controller.

The assembly makes it possible to produce a heat of approximately 70° C. and rapid heat transfer.

Removed from the oven, the eyelashes are clasped in said assembly which is then cooled for 5 minutes, then demoulded.

The additional fibres are attached to the eyelashes and the connection is aesthetic.

An attachment without overlap is obtained, as illustrated in FIG. 15B.

### Example 7: Assembly Containing Additional Fibres

The cosmetic composition and the device are the same as those used in Example 1.

Additional fibres 1 cm in length are placed in the imprints of one of the two parts of the mould, as illustrated in FIG. 16A.

The composition is melted and then one drop of the composition is placed, with a hot micropipette, at each end of the additional fibres, as represented in FIG. 16B.

After cooling of the composition, one part of the mould thus prepared is mounted on one jaw and one part of the unused mould is mounted on the other jaw.

Before clasping the eyelashes, the jaws and the mould are heated for 5 minutes.

The eyelashes are then clamped and the assembly is heated for 2 minutes.

The assembly is left to cool for 5 minutes, then demoulded.

The additional fibres are attached to the eyelashes and the connection is aesthetic.

An attachment with overlap is obtained, as illustrated in FIG. 15A. Indeed, the preheating melts each of the small drops of composition and, in doing so, allows the eyelashes to advance towards the additional fibres.

### Example 8: Assembly Containing Additional Fibres

A device identical to that of Example 1 is produced, with the difference that the elastomeric mould comprises, in addition to the grooves, a groove which is 1 mm deep across a width of 4 mm and transverse to the length of the mould. The transverse groove is produced 5 mm from the edge.

Additional fibres are, as in Example 7, placed in the grooves. The meltable composition, as described in Example 1, is placed in the transverse groove, then the whole thing is heated in order to melt the composition, and then left to cool.

While cooling, the material traps the additional fibres along the width of the groove. The melted then cooled composition is mainly on the part of the additional fibres facing the transverse groove. There is also a small amount of melted then cooled composition along the length of the additional fibres (along a length of approximately 5 mm).

Finally, a jaw-comprising heating system as described in Example 7 is used to weld the additional fibres to the existing eyelashes.

### Examples 9 and 10: Fluid Composition Containing Particles in the Dispersed State

### Example 9: Assembly Containing a Composition Based on Wax and Latex and a Device which has a Heating Element

1) Solidifiable Cosmetic Composition

A composition in accordance with the invention is prepared using the weight proportions described below.

The composition is fluid at ambient temperature.

| | % by weight (relative |
|---|---|
| | to the total weight of |
| Compounds | the composition) |
| Aqueous microdispersion of carnauba wax sold | 50 |
| under the name Mexoryl SAP by the | |
| company Chimex | |
| Latex Styrene/Acrylate/Ammonium Methacrylate | 50 |
| copolymer sold under the name Syntran 5760 | |
| CG by the company Interpolymer | |

2) Device

The device used in Example 9 is as described in FIGS. 1A and 1B.

It contains two mobile jaws (3a) and (3b), one of the two jaws having a temperature-controlled heating system.

The two parts (2a) and (2b) of the mould, placed between the two jaws, are made of crosslinked silicone elastomer and are 2 mm thick. They each comprise 20 grooves approximately 0.5 mm in diameter over a length of 2.5 cm.

The two jaws comprise two lugs and the two parts of the mould each comprise two housings which fit the lugs.

When the two parts of the mould are brought together, 20 leaktight cavities, closed at their end, are created on the sides.

3) Cosmetic Assembly

The solidifiable cosmetic composition based on wax and latex is placed in the cavities and grooves of the two parts (2a) and (2b) of the mould.

The two jaws (3a) and (3b) containing the two parts (2a) and (2b) of the mould are moved near to the eyelash fringe and then closed so as to trap it.

The heating is activated by virtue of the heating element of the device (1) for 5 minutes until a temperature of approximately 70° C. is reached.

The heating is then stopped and the assembly is left to cool for 10 minutes. The two jaws (3a) and (3b) are then opened. The cosmetic composition has become solid by aggregation of the particles during the increase in temperature.

The two parts (2a) and (2b) of the mould remain attached to the eyelash fringe. The two parts of the mould are therefore moved apart, by pulling along their width, in order to release the eyelashes.

The result is then slightly rubbed in order to break the possible bridges of composition formed between two imprints.

A homogeneous makeup result is obtained on the eyelashes. The eyelashes are coated with a smooth, homogeneous and cohesive deposit.

### Example 10: Assembly Containing a Composition Based on Latex and Ethanol and a Device which has a Heating Element

1) Solidifiable Cosmetic Composition

A composition in accordance with the invention is prepared using the weight proportions described below.

The composition is fluid at ambient temperature.

| | % by weight (relative |
|---|---|
| | to the total weight of |
| Compounds | the composition) |
| Latex Polyester-5 sold under the name Eastman | 40 |
| AQ 55 S Polymer by the company Eastman | |
| Chemicals | |
| Ethanol | 20 |
| Water | qs 100 |

2) Device

The device used is identical to that of Example 9.

3) Cosmetic Assembly

The solidifiable cosmetic composition based on latex and ethanol is placed in the cavities and grooves of the two parts (2a) and (2b) of the mould.

The two jaws (3a) and (3b) containing the two parts (2a) and (2b) of the mould are moved near to the eyelash fringe and then closed so as to trap it.

The heating is activated by virtue of the heating element of the device (1) for 5 minutes until a temperature of approximately 70° C. is reached.

The heating is then stopped and the assembly is left to cool for 10 minutes. The two jaws (3a) and (3b) are then opened. The cosmetic composition has become solid by aggregation of the latex particles during the increase in temperature.

The two parts (2a) and (2b) of the mould remain attached to the eyelash fringe. The two parts of the mould are therefore moved apart, by pulling along their width, in order to release the eyelashes.

The result is then slightly rubbed in order to break the possible bridges of composition formed between two imprints.

A homogeneous makeup result is obtained on the eyelashes. The eyelashes are coated with a smooth, homogeneous and cohesive deposit.

### Examples 11 to 16: Fluid Composition Comprising an Activatable Compound

### Example 11: Assembly Containing a Composition Based on Methacrylate Compounds

1) Cosmetic Composition

A composition in accordance with the invention is prepared. The formula consists of 100% by weight of methacrylate compounds sold under the name Power Polish™ Top Coat by the company CND Shellac. The composition is handled while taking care to avoid contact with daylight.

2) Device

The device used in Example 11 is as described in FIGS. 1A and 1B.

The two parts of the mould (2a) and (2b), placed between two mobile jaws (3a) and (3b), are made of UV-transparent silicone elastomer (Platsil gel 10 (A:B) reactive silicones sold by the company Polytek). They each comprise 20 grooves approximately 0.5 mm in diameter over a length of 2.5 cm.

The two jaws comprise two lugs and the two parts of the mould each comprise two housings which fit the lugs.

When the two parts of the mould are brought together, 20 leaktight cavities, closed at their end, are created on the sides.

3) Cosmetic Assembly

The cosmetic composition based on methacrylate compounds is placed in the grooves of the lower part (2b) of the mould. The upper part (2a) of the mould is then put in place.

The two parts (2a) and (2b) of the mould are then placed on the jaws (3a) and (3b) of the device (1).

The two jaws (3a) and (3b) are moved near to the eyelash fringe and then closed so as to trap it.

The assembly is placed in proximity to a UVA source (UV lamp used for UV gels producing 36 W).

After 4 minutes of exposure, the two jaws are opened and the sculptures are released.

A homogeneous makeup result is obtained on the eyelashes, without any eyelash brushing movement. The makeup is easy to apply without any need to calibrate the amount of composition taken and applied.

### Example 12: Assembly Containing a Reactive Composition Based on Ethyl Cyanoacrylate

1) Cosmetic Composition

A composition in accordance with the invention is prepared. The formula consists of 100% by weight of ethyl cyanoacrylate sold under the name Superglue 3 by the company Loctite. The composition is handled while taking care to avoid contact with water.

2) Device

The device used is identical to that of Example 11.

3) Cosmetic Assembly

The cosmetic composition based on ethyl cyanoacrylate is placed in the grooves of the lower part (2b) of the mould. The upper part (2a) of the mould is moistened by spraying so as to deposit thereon 0.3 g of water, and then put in place.

The two parts (2a) and (2b) of the mould are then placed on the jaws (3a) and (3b) of the device (1).

The two jaws (3a) and (3b) are moved near to the eyelash fringe and then closed so as to trap it.

After waiting for 4 minutes, the two jaws are opened and the sculptures are released.

A homogeneous makeup result is obtained on the eyelashes, without any eyelash brushing movement. The makeup is easy to apply without any need to calibrate the amount of composition taken and applied.

### Example 13: Assembly Containing a Composition Based on a Reactive Silicone

1) Cosmetic Composition

A composition in accordance with the invention is prepared. The formulation consists of 100% by weight of a reactive silicone sold under the name Joint & Fix by the company GEB. The composition is handled while taking care to avoid contact with water.

2) Device

The device used is identical to that of Example 11.

3) Cosmetic Assembly

The cosmetic composition based on a reactive silicone is placed in the grooves of the lower part (2b) of the mould. The upper part (2a) of the mould is moistened by spraying so as to deposit thereon 0.1 g of water, and then put in place.

The two parts (2a) and (2b) of the mould are then placed on the jaws (3a) and (3b) of the device (1).

The two jaws (3a) and (3b) are moved near to the eyelash fringe and then closed so as to trap it.

After waiting for 12 minutes, the two jaws are opened and the sculptures are released.

A homogeneous makeup result is obtained on the eyelashes, without any eyelash brushing movement. The makeup is easy to apply without any need to calibrate the amount of composition taken and applied.

### Example 14: Assembly Containing a Composition Based on Reactive Silicones

1) Cosmetic Composition

A composition in accordance with the invention is prepared. The formulation consists of two components of Platsil gel 10 (A:B) reactive silicones sold by the company Polytek.

The two compounds are mixed and the mixture is introduced into the mould without delay, less than 3 minutes after preparing the mixture.

2) Device

The device used is identical to that of Example 11.

3) Cosmetic Assembly

The cosmetic composition based on reactive silicones is placed, immediately after it has been prepared, in the grooves of the lower part (2b) of the mould. The upper part (2a) of the mould is then put in place.

The two parts (2a) and (2b) of the mould are then placed on the jaws (3a) and (3b) of the device (1).

The two jaws (3a) and (3b) are moved near to the eyelash fringe and then closed so as to trap it.

After waiting for 7 minutes, the two jaws are opened and the sculptures are released.

A homogeneous makeup result is obtained on the eyelashes, without any eyelash brushing movement. The makeup is easy to apply without any need to calibrate the amount of composition taken and applied.

### Example 15: Assembly Containing a Composition Based on Reactive Silicones

1) Cosmetic Composition

A composition in accordance with the invention is prepared. The formulation consists of two components of Platsil gel 10 (A:B) reactive silicones sold by the company Polytek. The mixing of the components is carried out in situ in the device.

2) Device

The device used is identical to that of Example 11.

3) Cosmetic Assembly

The component A is placed in the grooves of the lower part (2b) of the mould. As regards the compound. B, it is placed in the upper part (2a) of the mould.

The two parts (2a) and (2b) of the mould are then placed on the jaws (3a) and (3b) of the device (1).

The two jaws (3a) and (3b) are moved near to the eyelash fringe and then closed so as to trap it.

After waiting for 7 minutes, the two jaws are opened and the sculptures are released.

A homogeneous makeup result is obtained on the eyelashes, without any eyelash brushing movement. The makeup is easy to apply without any need to calibrate the amount of composition taken and applied.

### Example 16: Assembly Containing a Composition of Sodium Alginate and Calcium Chloride

1) Cosmetic Composition

A composition in accordance with the invention is prepared as described below.

The formulation consists of, on the one hand, a solution of sodium alginate (sold under the name Kelcosol by the company ISP) at 5% and, on the other hand, of an aqueous solution of calcium chloride at 3%, having a pH=7, gelled with a non-ionic hydroxyethylcellulose thickener at 2%. The compositions are prepared by dissolving the compounds in water.

The mixing of the components is carried out in situ in the device.

2) Device

The device used is identical to that of Example 11.

3) Cosmetic Assembly

The solution based on sodium alginate is placed in the grooves of the lower part (2b) of the mould. As regards the gelled solution based on calcium chloride, it is placed, in an amount equivalent to the amount of alginate solution, in the upper part (2a) of the mould.

The two parts (2a) and (2b) of the mould are then placed on the jaws (3a) and (3b) of the device (1).

The two jaws (3a) and (3b) are moved near to the eyelash fringe and then closed so as to trap it.

After waiting for 3 minutes, the two jaws are opened and the sculptures are released.

A homogeneous makeup result is obtained on the eyelashes, without any eyelash brushing movement. The makeup is easy to apply without any need to calibrate the amount of composition taken and applied.

### Examples 17 to 19: Ductile Composition

### Example 17

1) Cosmetic Composition

A composition in accordance with the invention is prepared using the weight proportions described below.

| | % by weight (relative to |
|---|---|
| | the total weight of the |
| Compounds | composition) |
| Carnauba wax sold under the name | 3.2 |
| Copernicia cerifera waw by the | |
| company Bauerlocher | |
| Beeswax sold under the name Cera Alba | 4.1 |
| by the company Strahl & Pitsch | |
| Paraffin wax sold under the name Paraffine | 12.9 |
| raffinée [Refined paraffin] by the company | |
| Avel | |
| Hydroxyethylcellulose sold under the name | 0.9 |
| Hydroxyethylcellulose by the company | |
| Amerchol | |
| Gum arabic sold under the name Acacia | 3.4 |
| Senegal by the company Alland & Robert | |
| Iron oxide | 7.1 |
| Preservative | 1 |
| Cetyl phosphate sold under the name Arlatone | 6.7 |
| MAP by the company Uniqema | |
| Steareth-2 sold under the name Brij 72 by the | 3.3 |
| company Uniqema | |
| Water | qs 100 |

2) Device

The device used in Example 17 is as described in FIGS. 1A and 1B.

The two parts of the mould (2a) and (2b), placed between two mobile jaws (3a) and (3b), are made of silicone elastomer (Platsil gel 10 (A:B) reactive silicones sold by the company Polytek). They each comprise 20 grooves approximately 0.5 mm in diameter over a length of 2.5 cm.

The two jaws comprise two lugs and the two parts of the mould each comprise two housings which fit the lugs.

When the two parts of the mould are brought together, 20 leaktight cavities, closed at their end, are created on the sides. The volume of these cavities can be reduced by 25% or more.

Before compression, the volume of each cavity is equal to 370 mm³. Under compression, the mould deforms and pushes the cavities to be reduced to a volume of 250 mm³.

3) Cosmetic Assembly

Vegetable oil and boron nitride powder in the form of platelets (Boron Nitride from the company Merck) are placed, in a proportion of respectively 25 mg and 50 mg, in each of the parts (2a) and (2b) of the mould.

The cosmetic composition described above is then placed in the grooves of the two parts (2a) and (2b) of the mould, until they are filled.

The composition is left to dry for 10 minutes.

The two jaws (3a) and (3b) containing the two parts (2a) and (2b) of the mould are moved near to the eyelash fringe and then closed so as to trap it.

The eyelashes are strongly compressed, by applying a pressure of approximately 4 kg on the upper jaw for 1 minute.

The two jaws are then opened and the sculptures are released.

A homogeneous makeup result is obtained on the eyelashes. The layer of mascara has taken the shape of the mould.

### Example 18

1) Cosmetic Composition

A composition in accordance with the invention is prepared using the weight proportions described below by dissolving the sulfonic polyester in ethanol.

| | % by weight (relative to |
|---|---|
| | the total weight of the |
| Compounds | composition) |
| Sulfonic polyester sold under the name sulfonic | 60 |
| polyester AQ 1350 by the company | |
| Eastman Chemicals | |
| Ethanol | 40 |

2) Device

The device used is identical to that of Example 17.

3) Cosmetic Assembly

Vegetable oil and boron nitride powder in the form of platelets (Boron Nitride from the company Merck) are placed, in a proportion of respectively 25 mg and 50 mg, in each of the parts (2a) and (2b) of the mould.

The cosmetic composition described above is then placed in the grooves of the two parts (2a) and (2b) of the mould, until they are filled.

The composition is left to dry for 24 hours.

The two jaws (3a) and (3b) containing the two parts (2a) and (2b) of the mould are moved near to the eyelash fringe and then closed so as to trap it.

The eyelashes are strongly compressed, by applying a pressure of approximately 4 kg on the upper jaw for 1 minute.

The two jaws are then opened and the sculptures are released.

A homogeneous makeup result is obtained on the eyelashes. The layer of mascara has taken the shape of the mould.

### Example 19

1) Cosmetic Composition

A composition in accordance with the invention is prepared. It consists of Chavant modelling clay sold by the company Esprit composite under the name Plastiline Ivoire.

2) Device

The device used is identical to that of Example 17.

3) Cosmetic Assembly

Vegetable oil and boron nitride powder in the form of platelets (Boron Nitride from the company Merck) are placed, in a proportion of respectively 25 mg and 50 mg, in each of the parts (2a) and (2b) of the mould.

The cosmetic composition described above is then placed in the grooves of the two parts (2a) and (2b) of the mould, until they are filled.

The two jaws (3a) and (3b) containing the two parts (2a) and (2b) of the mould are moved near to the eyelash fringe and then closed so as to trap it.

The eyelashes are strongly compressed, by applying a pressure of approximately 4 kg on the upper jaw for 1 minute.

The two jaws are then opened and the sculptures are released.

The material has integrated the eyelashes, while taking the shape of the mould.

The figures and the examples were described with eyelashes as keratin fibres, but these figures and examples also apply to other types of keratin fibres, in particular to eyebrows or the hair.

The implementation particularities of the various examples described can be combined within variants which are not illustrated.

The expression “comprising a” should be understood as being synonymous with “comprising at least one”, unless otherwise specified.

1-43. (canceled)

44. Process for cosmetic treatment of one or more keratin fibres, comprising the moulding of at least one cosmetic composition onto at least one part of said fibre(s), by means of one or more cavities of a mould into which said fibre(s) is (are) at least partially introduced.

45. Process according to claim 44, the composition having a melting point of between 40° C. and 120° C., and comprising at least 15% by weight of meltable compound(s), relative to the total weight of the composition, and being brought to a temperature greater than or equal to its melting point.

46. Process according to claim 44, the composition being fluid at ambient temperature and containing at least particles in the dispersed state in a liquid medium, said composition being solidifiable via the aggregation of said particles.

47. Process according to claim 44, the composition being fluid at ambient temperature and containing at least one compound that is activatable by a physical or chemical stimulus, said composition being solidifiable via the conversion of the activated form of said compound into a solid material, by self-reaction and/or interaction with at least one supplementary compound.

48. Process according to claim 44, the composition being ductile.

49. Process according to claim 44, the cavity or cavities of the mould being formed by bringing two jaws together.

50. Process according to claim 44, the composition being moulded onto at least one part of at least one of said keratin fibres and of at least one additional fibre, the composition ensuring the attachment of the additional fibre(s) to the keratin fibre(s), with or without axial overlap between a keratin fibre and an additional fibre.

51. Assembly comprising at least one cosmetic composition for cosmetic treatment of keratin fibres and a device comprising a mould, the mould comprising at least one cavity into which or each of which at least one of said fibres can be at least partially introduced, in such a way that the composition is moulded onto at least one part of said fibre(s) present in the cavity or cavities.

52. Assembly according to claim 51, the cavity or cavities being formed by bringing two jaws together.

53. Assembly according to claim 51, the cavity or cavities each having a length of between 5 and 30 mm and a width of between 150 μm and 3 mm.

54. Assembly according to claim 51, each cavity having an elongated shape and being closed at at least one of its longitudinal ends.

55. Assembly according to claim 51, the device comprising at least one heating element which serves to increase the temperature of the composition.

56. Assembly according to claim 51, the device comprising a system which assists the drying of the composition, the system providing energy and/or comprising an aeration circuit, suction and/or blowing of air.

57. Assembly according to claim 51, the device comprising at least one system for admitting material, which serves to introduce a part or all of the composition into the mould cavity or cavities.

58. Assembly according to claim 51, the device comprising at least one light element or microwave element.

59. Assembly according to claim 51, the device comprising blades or other reliefs which are used to cut bridges of composition between at least two cavities after moulding.

60. Assembly according to claim 51, the device comprising one or more additional fibres, the composition ensuring the attachment of the additional fibre(s) to the keratin fibre(s), with or without axial overlap between a keratin fibre and an additional fibre.

61. Assembly according to claim 52, the device being in the form of a clamp comprising at least one housing into which at least one of the fingers of one hand can be introduced so as to move the two jaws apart.

62. Assembly according to claim 52, the device being in the form of a clamp comprising two housings into which two of the fingers of one hand can be introduced so as to move the two jaws apart.

63. Assembly according to claim 51, the mould comprising at least two parts, one part comprising one or more imprints, each of the imprints being of rounded cross section, the other part being devoid of imprint, so as to form the cavity or cavities of the mould when the parts of the mould are brought together.

64. Assembly according to claim 51, the mould comprising at least two parts, each of two parts comprising one or more imprints, the imprint(s) of one of the parts being placed opposite the imprint(s) of at least one other part, so as to form the cavity or cavities of the mould when the parts of the mould are brought together.

65. Assembly according to claim 51, each imprint having a depth of between 75 μm and 1.5 mm.

66. Assembly according to claim 51 each cavity constituting a space which is substantially entirely closed in the absence of keratin fibre.

67. Assembly according to claim 51, each cavity delimiting a space which is closed with the exception of one end via which the keratin fibre(s) which are at least partially introduced into the cavity communicate with the exterior.
